(19) United States
(12) Patent Application Publication
(10) Pub. No.: US 2016/0243080 A1
(43) Pub. Date: Aug. 25, 2016

(54) FORMULATIONS OF ANGIOTENSIN RECEPTOR BLOCKERS

(71) Applicants: Peter M. Abadir, Woodstock, MD (US); Jeremy D. Walston, Baltimore, MD (US)

(72) Inventors: Peter M. Abadir, Woodstock, MD (US); Jeremy D. Walston, Baltimore, MD (US)

(21) Appl. No.: 15/042,865

(22) Filed: Feb. 12, 2016

Related U.S. Application Data
(60) Provisional application No. 62/190,038, filed on Jul. 8, 2015.
(60) Provisional application No. 62/116,065, filed on Feb. 13, 2015.

(51) Int. Cl. A61K 31/41; A61K 47/38; A61K 47/12; A61K 47/32; A61K 47/02; A61K 9/00; A61K 9/06

(52) U.S. Cl. CPC A61K 31/41; A61K 9/0014; A61K 47/38; A61K 9/06; A61K 47/32; A61K 47/02; A61K 47/12

(57) ABSTRACT

Provided herein are pharmaceutical compositions for the treatment of wounds, including chronic wounds and diabetic ulcers. The pharmaceutical compositions, which comprise valsartan, inhibit angiotensin receptors in the wound bed. Also provided herein are methods of making the pharmaceutical compositions of the invention, and methods for treating wounds in patients in need thereof.

### RELATED APPLICATIONS

This application claims the benefit of priority to U.S. Provisional Patent Application Ser. No. 62/116,065, filed Feb. 13, 2015 and U.S. Provisional Patent Application Ser. No. 62/190,038, filed Jul. 8, 2015, the contents of which are hereby incorporated by reference.

### BACKGROUND

Chronic wounds are among the most common, painful, debilitating and costly conditions in diabetics and older adults, and are frequently a portal for bacterial infections that can lead to amputations, sepsis, and mortality. Most current chronic wound care treatments are technologies that target infections or that debride necrotic tissue. Others focus on the use of skin substitutes, biologic wound products such as growth factors, or hyperbaric oxygen as an adjunct in wound healing.

The biology of normal wound healing includes sequential yet overlapping inflammatory, proliferative, and remodeling phases that involve complex biological signaling. Dysregulation of this signaling is thought to underlie skin breakdown, poor healing and the development of chronic wounds. The renin angiotensin system (RAS) is a hormonal system that is involved in various stages of wound healing. RAS is involved in the inflammatory response, collagen deposition, and in tissue-related growth factor (TGF-β) signaling involved in wound healing. In aging patients, and in patients suffering from diabetes, RAS is dysregulated, having increased expression of the pro-inflammatory angiotensin II type 1 receptor (AT₁R) and decreased expression of the pro-inflammatory angiotensin II type 2 receptor (AT₂R), which may play a role in skin vulnerability associated with aging and diabetes. An increase the AT₁R expression can lead to thinning of the epidermis, degeneration of collagen, fracture of the dermal layer, and atrophy of subcutaneous fat. Previously, some studies have focused on the use of angiotensin II receptor agonists during early stages of wound healing.

There exists a need to develop therapies for wound healing that target the dysregulated renin angiotensin system in diabetic and older patients.

### SUMMARY OF THE INVENTION

The invention relates to pharmaceutical compositions for the treatment of wounds, including chronic wounds and diabetic ulcers. The pharmaceutical compositions, which comprise valsartan, inhibit angiotensin receptors in the wound bed. In another aspect, the invention provides methods of making the pharmaceutical compositions of the invention, and methods for treating wounds in patients in need thereof.

### BRIEF DESCRIPTION OF THE DRAWINGS

FIG. 1 shows the results of a toxicity study in which valsartan was quantified in porcine plasma.

FIG. 2 is shows a standard curve in which known concentrations of valsartan were spiked into porcine plasma to generate standards (black circles) and quality control samples (blue triangles).

FIG. 3 is a schematic diagram of wound site design.

FIG. 4 shows representative photographs from study days 29 and 42. Left Side: Treatment B (1% valsartan composition), Right Side: Treatment C (Placebo).

FIG. 5 is a graph showing healing rates for combined animal data (means).

FIG. 6 contains graphs demonstrating healing rates for individual animals.

FIG. 7 is a bar graph showing the wound gap measured from wounds treated with the 1% valsartan formulation versus the placebo formulation.

FIG. 8 is a bar graph showing the epithelial grade measured from wounds treated with the 1% valsartan formulation versus the placebo formulation.

FIG. 9 is a bar graph showing the epithelial thickness measured from wounds treated with the 1% valsartan formulation versus the placebo formulation.

FIG. 10 is a bar graph showing the dermal thickness measured from wounds treated with the 1% valsartan formulation versus the placebo formulation.

FIG. 11 is a bar graph showing the effect of various dosages of valsartan in wound healing (as a measure of wound size).

FIG. 12 is a graph showing a plannimetric assessment of wound closure rate in diabetic (Leprdb) mice treated with different doses of Valsartan and Losartan gels applied 7 days after wounding.

FIG. 13 is a bar graph showing the effect 1% valsartan versus 1% losartan in wound healing (as a measure of wound size).

FIG. 14 shows a Kaplan Meier analysis of complete wound closure of Leprdb mice treated with 1% Valsartan.

FIG. 15 is a graph showing the effect of various wound treatment regimens on wound healing.

FIG. 16 consists of panels A and B. Panel A shows a comparison between 1% Valsartan gel and 5% Captopril gel demonstrating delayed healing with Captopril. Panel B shows that 1% valsartan gel failed to accelerate wound closure in AT₂R^{-/-} mice.

FIG. 17 is a graph showing increased collagen deposition in wounds of Leprdb mice with valsartan gel as compared to placebo, Captopril (CAP), or the combination of valsartan and captopril (CAP+VAL).

FIG. 18 is a bar graph showing a comparison of wound healing efficacy in ointment formulations versus gel formulations.

FIG. 19 consists of panels A-D and shows wound closure measurements in aged diabetic pigs treated with daily 1% Valsartan gel. Panel A shows representative images and panel B shows a plannimetric assessment of changes in wound size in aged diabetic pigs treated with 1% Valsartan gel. Longitudinal Tissue composition analysis of porcine wounds was conducted (panels D and E) to identify different components of wounds and to track changes in epithelization (Panel D) and slough (Panel E) longitudinally. Data shows higher epithelization and clearance of slough in Valsartan treated wounds. Data are means±SEM. ****p<0.0001.

FIG. 20 shows changes in the TGF superfamily downstream signaling proteins in wounds of aged diabetic pigs. Valsartan treated wounds have higher expression of Smad3, phosphorylated Smad3 and the common mediator Smad4 in healed skin as compared to placebo. A decrease in the expression of Smad1, Smad2 and phosphorylated Smad1, 5 and 9 Smad3 was also observed in the Valsartan treated wounds. The photomicrographs presented in red or green fluorescent staining with a blue DAPI counter stain for nuclei at 63× magnification. Quantification of the levels of Smads in porcine wounds is shown. Scale bar 200 μm. Data are mean fluorescence intensity±SEM. ****p<0.0001.

FIG. 21 is a series of images demonstrating enhanced mitochondrial, proliferation and angiogenesis markers in aged diabetic pig wounds treated with Valsartan. Higher mitochondrial Cox IV was seen in wounds treated with daily Valsartan gel. Treated wounds also exhibited higher actin (α-SMA), increased phosphorylation of p42/44 MAPK and vascular endothelial growth factor (VEGF) receptor2. The photomicrographs presented in red or green fluorescent staining with a blue DAPI counter stain for nuclei at 63× magnification. Quantification of the levels of Smads in porcine wounds is shown. Scale bar 200 μm. Data are mean fluorescence intensity±SEM. ****p<0.0001.

FIG. 22 consists of panels A-K and is a series of images showing Masson's trichrome and hematoxylin and eosin (H&E) staining of skin sections from aged diabetic pigs shows an expanded zone of dermal collagen with valsartan treatment. Low magnification (4×) of Valsartan (panel A) and Placebo (Panel B) treated wounds showing marked difference in total thickness (scale bar 4 mm). Representative image of epidermal layers in Valsartan (Panel C) and placebo (panel D) treated healing skin (40×. Scale bar 50 um). Representative image showing collagen arrangement in Valsartan (Panel E) and placebo (Panel F) treated healing skin (40×. Scale bar 50 um). Quantification of the thickness of the zones of epidermis and dermal collagen in porcine wounds shown. H&E image analysis demonstrate more frail healing in placebo treated wounds. Biomechanical assessment of healed skin in pig cohorts (Panels J and K). Comparison of the average tension at the breaking point of pig groups (Panel J) and average work at the breaking point (Panel K) of both groups. Data are means±SEM. ***p<0.001.

### DETAILED DESCRIPTION OF THE INVENTION

The present invention is based on the surprising discovery that inhibition of the angiotensin II type 1 receptor during the proliferative/remodelling phase of wound healing results in enhanced wound repair in diabetic mice. The formulations described herein are a unique approach to wound management due to their focus on the blockade of angiotensin receptor blockers in the skin. The formulations of the invention, in use, specifically block these receptors in the wound bed, targeting the proliferative/remodelling phases of wound healing.

The biology of normal wound healing includes sequential yet overlapping inflammatory, proliferative, and remodelling phases that involve complex biological signaling. Dysregulation of specific signaling pathways is thought to underlie skin breakdown and poor wound healing. Most new wound treatments have not targeted these phases and pathways, but instead have targeted infections and debridement.

The renin angiotensin system is active in connective tissue and skin, and is known to be important in wound healing. RAS is involved in the inflammatory response, collagen deposition and in tissue-related growth factor (TGF-β) signaling necessary for wound healing. RAS is known to be dysregulated in both aging and in diabetes, with increased AT₁R and decreased angiotensin II type 2 receptor (AT₂R) expression in diabetic wound healing and in aging, which may play a role in aging skin vulnerability. Indeed, an altered dermal AT₁R and AT₂R ratio is associated with thinning of epidermis, degeneration of collagen, fracture of dermal layer, and atrophy of subcutaneous fat in diabetic rats. These changes are consistent with those seen in aging skin. The inventors and others have demonstrated that losartan can modify the AT₁R and AT₂R ratio, and can greatly accelerate skeletal muscle healing in older mice.

Working in diabetic mouse and pig models, the inventors surprisingly discovered that application of 1% valsartan ointment to a wound, the 1% valsartan treatment starting 7 days after the initial formation of the wound (i.e., in the proliferative phase of wound healing) significantly accelerated time to wound closure and improved tensile strength of treated skin, as compared to animals treated at other time points (e.g., in the inflammatory phase of wound healing) or with control ointment. Also surprisingly, the application of topical valsartan during the inflammatory phase (e.g., in the first 1-7 days after wounding) significantly impaired wound healing.

### Pharmaceutical Compositions

In certain embodiments, the present invention provides a topical pharmaceutical composition, comprising valsartan, in an amount from about 0.2% to about 2.5% by weight of the composition and a pharmaceutically acceptable carrier material. In certain embodiments, valsartan is present in the composition in an amount from about 0.5% to about 1.5%, about 0.6% to about 1.4%, about 0.7% to about 1.3%, about 0.75% to about 1.25%, about 0.8% to about 1.2%, or about 1% by weight of the composition. For ease of discussion, the topical pharmaceutical compositions described herein will be referred to as “1% valsartan composition”. It will be understood that reference to “1% valsartan”, “1% valsartan composition”, or “1% valsartan gel composition” can also refer to compositions having valsartan in an amount from about 0.2% to about 2.5% by weight, from about 0.5% to about 1.5%, about 0.6% to about 1.4%, about 0.7% to about 1.3%, about 0.75% to about 1.25%, about 0.8% to about 1.2%, or about 1% by weight of the pharmaceutical composition.

In certain embodiments, the pharmaceutical composition comprises a pharmaceutically acceptable carrier material that is a cellulosic gel. In certain embodiments, the cellulosic gel is present in an amount of about 5% to about 99% of the composition. In certain embodiments, the cellulosic gel is present in the composition in an amount from about 20% to about 99%, about 30% to about 99%, about 40% to about 99%, about 50% to about 99%, about 50% to about 98%, about 60% to about 99%, about 60% to about 98%, about 70% to about 98%, about 75% to about 98%, about 80% to about 98%, about 85% to about 98%, about 85% to about 97%, about 90% to about 98%, about 90% to about 97%, about 92% to about 99%, about 92% to about 98%, about 92% to about 97%, about 93% to about 99%, about 93% to about 98%, about 93% to about 97%, about 94% to about 97%, about 94% to about 96%, or about 95% by weight of the pharmaceutical composition.

In certain embodiments, the pharmaceutical composition further comprises an aqueous medium, for example, water, or saline. The aqueous medium may be present in the composition in an amount from about 1% to about 5% by weight of the composition. In certain embodiments, the aqueous medium is present in an amount of about 1.5% to about 4.5%, about 2% to about 4%, about 2.5% to about 3.5%, or about 3% of the composition.

In certain embodiments, the cellulosic gel comprises hydroxypropyl methylcellulose, hydroxypropylcellulose, methylcellulose, or a combination thereof. In certain embodiments, the cellulosic gel comprises hydroxypropylmethylcellulose. In further embodiments, the cellulosic gel further comprises propylene glycol, polypropylene glycol, chlorhexidine (e.g., chlorhexidine gluconate), water, propylene oxide, acetic acid, sodium acetate, and fragrance. In certain embodiments, the fragrance is lavender. In certain embodiments, the cellulosic gel comprises hydroxypropyl methylcellulose and propylene glycol in a weight ratio of about 1 to 3.

In some embodiments, the cellulosic gel is Surgilube®.

In certain embodiments, the cellulosic gel confers certain anti-bacterial properties to the composition. For example, chlorhexidine (e.g., chlorhexidine gluconate) is an anti-bacterial that can be used as an antiseptic for applications to wounds.

In certain embodiments, the cellulosic gel comprises one or more anti-bacterial agents.

In certain embodiments, the cellulosic gel comprises glycerin and hydroxyethyl cellulose. In further embodiments, the cellulosic gel comprises glycerin, hydroxyethyl cellulose, chlorhexidine (e.g., chlorhexidine gluconate), glucolactone (e.g., glucono delta-lactone), methylparaben, and sodium hydroxide. In certain embodiments, the cellulosic gel is K-Y® Jelly.

In certain embodiments, the pharmaceutical composition further comprises crospovidone, hydroxypropyl methylcellulose, ferric oxide, magnesium stearate, and titanium dioxide.

In certain embodiments the pharmaceutical composition further comprises colloidal silicon dioxide, crospovidone, hydroxypropyl methylcellulose, ferric oxide, magnesium stearate, microcrystalline cellulose, polyethylene glycol, and titanium dioxide.

In certain embodiments, the pharmaceutical composition further comprises cellulose compounds, crospovidone, gelatin, ferric oxide, magnesium stearate, povidone, sodium lauryl sulfate, and titanium dioxide.

In certain embodiments, the pharmaceutical composition of the invention consists essentially of valsartan, in an amount from about 0.2% to about 2.5% by weight of the composition; colloidal silicon dioxide, crospovidone, hydroxypropyl methylcellulose, ferric oxide, magnesium stearate, microcrystalline cellulose, polyethylene glycol, titanium dioxide, propylene glycol, polypropylene glycol, chlorhexidine gluconate, water, propylene oxide, acetic acid, sodium acetate, and lavender.

In certain embodiments, the pharmaceutical composition of the invention comprises valsartan, in an amount of about 1% by weight of the composition, hydroxypropyl methylcellulose, in an amount of about 23% to about 24% by weight of the composition, and propylene glycol, in an amount of about 71% to about 72% by weight of the composition.

In certain embodiments, the composition includes one or more anti-bacterial agents, anti-microbial agents, anti-scarring agents, permeation enhancers, growth factors, and anesthetics. For example, the composition may comprise chlorhexidine.

In certain embodiments, the specific gravity range for the compositions of the invention is about 0.75 to about 1.1, about 0.8 to about 1, or about 0.9 at 20° C.

In certain embodiments, the viscosity range for the compositions of the invention is about 150 to about 1000 P.

In certain embodiments, the freezing point for the compositions of the invention is about −10° C. to about −20° C., about −12° C. to about −18° C., or about −15° C.

In certain embodiments, the boiling point for the compositions of the invention is about 100° C. to about 110° C., about 102° C. to about 108° C., or about 105° C.

In certain embodiments, the pH of the composition of the invention is about 4.0 to about 7.0, about 4.5 to about 6.5, or about 5.

In certain embodiments, the 1% valsartan formulation is a powder. In certain such embodiments, the pharmaceutically acceptable carrier material can be an alginate salt, such as calcium alginate or sodium alginate. Alginate salts such as calcium alginate may be prepared by methods known to persons of ordinary skill in the art.

Certain powder formulations are wet-to-dry mixes. In other words, the 1% valsartan powder formulation may be applied as a dry powder to a wound. Exposure of the powder to the wound exudate or, in certain embodiments, transudate, “activate” the powder, and convert the 1% valsartan formulation to a gel at the wound site.

In certain embodiments, the topical pharmaceutical composition of the invention is advantageous because topical, local administration avoids the systemic impact of valsartan, focusing the therapeutic effect of the drug on the local skin renin angiotensin system.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a valsartan and a pharmaceutically acceptable carrier. Pharmaceutically acceptable carriers can include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, the aqueous solution is pyrogen-free, or substantially pyrogen-free. The excipients can be chosen, for example, to effect delayed release of an agent or to selectively target one or more cells, tissues or organs.

In certain embodiments, the composition is a form suitable topical administration. The composition can also be present in a transdermal delivery system, e.g., a skin patch. The topically applicable form of the composition can a transdermal patch, ointment, cream, gel, suspension, liquid, elixir, or eye drop. Preferably, the topical composition is a gel, ointment, cream, bandage, spray, or powder.

In certain embodiments, the formulation is packaged as a pre-dosed formulation. For example, the formulation may include a tube for each day of wound treatment, wherein vehicle (e.g., cellulosic gel) is given in the first few days after wounding, then the formulation of the invention (i.e., 1% valsartan formulation) is administered during the days following. In such an example, the dosage is metered via a pre-dosed formulation such as a tube. Alternative, the pre-dosed formulation can be spray or droplets.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Though preferred carriers are described throughout, further examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Preferably, out of one hundred percent, this amount will range from about 0.2% to about 2.5%, about 0.5% to about 1.5%, about 0.6% to about 1.4%, about 0.7% to about 1.3%, about 0.75% to about 1.25%, about 0.8% to about 1.2%, or about 1% by weight of the composition.

Besides inert diluents, the oral compositions can also include adjuvants such as wetting agents, emulsifying and suspending agents, sweetening, flavoring, coloring, perfuming and preservative agents.

Formulations of the pharmaceutical compositions for administration to the mouth, e.g., buccal administration, may be presented as a mouthwash, or an oral spray, or an oral ointment.

Dosage forms for the topical or transdermal administration include powders, sprays, ointments, pastes, creams, lotions, gels, solutions, patches, bandages, inhalants, mouthwash, eye drops, and intranasal droplets. The active compound may be mixed under sterile conditions with a pharmaceutically acceptable carrier, and with any preservatives, buffers, or propellants that may be required.

The ointments, pastes, creams, lotions, and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day. In certain embodiments of the present invention, the composition may be administered two or three times daily, or as needed. In preferred embodiments, the composition will be administered once daily.

The patient receiving this treatment is any animal in need, including primates, in particular humans, and other mammals such as equines, cattle, swine and sheep; and poultry and pets in general.

### Methods for Preparing the 1% Valsartan Compositions

The present invention also provides methods for preparing the 1% valsartan composition. In certain embodiments, the methods of preparing the 1% valsartan composition comprise the step of combining a pharmaceutically acceptable carrier material with valsartan in an amount sufficient to make a composition that is 0.2% to about 2.5% valsartan by weight, 0.5% to about 1.5%, about 0.6% to about 1.4%, about 0.7% to about 1.3%, about 0.75% to about 1.25%, about 0.8% to about 1.2%, or about 1% by weight of the composition.

In certain embodiments, the valsartan used in preparing the 1% valsartan composition is a valsartan powder. In certain preferred embodiments, the valsartan powder contains no additional additives or fillers. In certain embodiments, the valsartan powder is of a purity of at least 90%, at least 95%, at least 96%, at least 97%, at least 98%, or at least 99% or greater.

In certain preferred embodiments, the pharmaceutically acceptable carrier (e.g., a cellulosic gel) is combined with a valsartan powder. Optionally, this step of combining occurs in the presence of an aqueous medium (e.g., water or saline).

In certain embodiments, the source of valsartan used in the methods of preparing the 1% composition is a valsartan tablet. In certain embodiments, the source of valsartan is a valsartan capsule. In certain embodiments, the methods of preparing the 1% valsartan composition further comprise crushing a valsartan tablet, thereby yielding a powder comprising valsartan.

The valsartan tablet can comprise an outer coating layer. In certain embodiments, the methods of making the 1% valsartan formulation further comprise removing the outer coating layer from a valsartan tablet. Removal of the coating layer can be achieved by wiping the tablet with, for example, a wet disposable cloth or wiper, or by dissolving the coating layer in a solubilizing medium. The tablet without the coating can then be dried. Removal of the coating layer can occur prior to crushing the tablet to yield a valsartan powder.

The valsartan powder obtained from crushing a tablet or capsule can further comprise any of the excipients originally present in the valsartan tablet or capsule. In certain embodiments, the amount of valsartan present in each tablet or capsule is known, and can be used to calculate the weight, or weight percent of valsartan in the valsartan powder.

In certain embodiments, the valsartan powder is combined with a liquid medium or an aqueous medium, for example water or saline, to form a mixture. This mixture can be combined with a pharmaceutically acceptable carrier material such as a cellulosic gel.

In certain other embodiments, the valsartan powder is combined simultaneously with a liquid medium or an aqueous medium, for example water or saline, and with a pharmaceutically acceptable carrier material such as a cellulosic gel.

In certain embodiments, the valsartan powder is combined with a pharmaceutically acceptable carrier material such as a cellulosic gel, then a liquid medium or an aqueous medium is added in an amount sufficient to reach desired consistency and specifications of the formulation.

Other protocols for combining the valsartan powder, the pharmaceutically acceptable material, and optionally the liquid medium would be known by a compounding pharmacist or other persons of ordinary skill in the art.

In certain embodiments, the cellulosic gel that is combined with the valsartan powder and aqueous medium mixture comprises hydroxypropyl methylcellulose, propylene glycol, polypropylene glycol, chlorhexidine gluconate, water, propylene oxide, acetic acid, sodium acetate, and lavender. In some embodiments, the cellulosic gel is Surgilube®.

In certain embodiments, the cellulosic gel that is combined with the valsartan powder and aqueous medium mixture comprises glycerin and hydroxyethyl cellulose. In further embodiments, the cellulosic gel comprises glycerin, hydroxyethyl cellulose, chlorhexidine (e.g., chlorhexidine gluconate), glucolactone (e.g., glucono delta-lactone), methylparaben, and sodium hydroxide. In certain embodiments, the cellulosic gel is K-Y® Jelly.

The cellulosic gel and the valsartan powder can be combined to generate a composition having a specific total weight or volume, and having a specific known weight percent of valsartan. Such a composition can be administered to a patient such that the amount of valsartan delivered to a wound site on the patient with each administration is known.

In general, the formulations are prepared by uniformly and intimately bringing into association valsartan with the pharmaceutically acceptable carrier material.

### Methods of Treatment

In certain embodiments, the invention relates to methods of treating a wound, comprising administering to a subject suffering from a wound a therapeutically effective amount of a 1% valsartan composition described herein.

In certain embodiments, the wound is a chronic wound, a diabetic skin ulcer, or is an ulcer associated with aging skin. In certain embodiments, the wound is a burn, an electrical injury, a radiation injury, a sunburn, a gun shot injury, an explosives injury, a post-surgical wound, a keloid, scar tissue, psoriasis, a superficial dermatologic resurfacing, or a skin lesion due to an inflammatory condition.

In certain embodiments, the step of administering is topical administration or buccal administration.

In certain embodiments, the pharmaceutical composition is administered at least 3 days, at least 4 days, at least 5 days, or at least 6 days after wounding.

In certain embodiments, the pharmaceutical composition is administered after the inflammatory phase of wound healing, or when the inflammatory phase of wound healing is coming to a conclusion. In certain embodiments, the pharmaceutical composition is administered during the proliferative and remodelling phases of wound healing.

Without being bound by theory, the data presented herein shows that the first inflammatory phase is critical for wound healing. Because valsartan is an anti-inflammatory compound, administration of valsartan during the inflammatory phase diminishes wound healing. Studies conducted by the inventors showed that administration of valsartan in the first few days made the wounds bigger.

The methods provided herein specifically target the proliferative and remodeling phases of wound healing. The data presented herein demonstrates a specific improvement in both mice and pigs when valsartan is applied during these phases. These results are based on the direct effects of valsartan on cell differentiation. The inflammatory phase generally lasts through the first days after injury. Administering valsartan slows or halts the abnormal chronic wound inflammatory phase often observed in diabetes and aging skin wounds, and triggers the proliferative phase. AT₁R blockade further enhances differentiation of the cells and positively impacts mitochondrial biology in the wound bed based on already known effects in literature.

Existing wound treatments are applied throughout the wound phases with no targeted biological specificity related to wound healing phase or mitochondrial function.

In certain embodiments, the subject is a mammal, for example a human.

The invention provides methods for treating a cutaneous wound, comprising administering to the cutaneous wound in a subject in need thereof a therapeutically effective amount of the 1% valsartan composition as described herein. In certain embodiments, the cutaneous wound is a chronic wound, a diabetic skin ulcer, or an ulcer associated with aging skin.

In certain embodiments, the cutaneous wound is in a tissue associated with an upregulation in angiotensin II type 1 receptors.

In certain embodiments, treatment of a wound may comprise applying to the wound a therapeutically effective amount of a 1% valsartan composition.

### Renin Angiotensin System in Skin and Its Role in Wound Healing

Several lines of evidence suggest that skin RAS activity plays a crucial role in most phases of wound healing. The results presented herein buttress prior reports on RAS effects on connective tissue healing and demonstrates efficacy of topical ARBs for chronic wound healing. Further, the results suggest that the beneficial effects of angiotensin system blockade seen with ARBs, does not extend to ACE inhibitors, implying a role for an un-opposed AT₂R in the acceleration of wound healing.

AT₁R amplifies inflammatory signaling, a necessary activating function that leads to proliferation phase, but one with potential negative consequences to wound healing in aging and diabetes as the inflammatory phase does not appropriately resolve enough to allow proliferation and remodeling in granulation tissue. The blockade of the AT₁R during early stages of wound healing was associated with a slower closure rate, perhaps resulting from the disruption of the inflammatory phase and impairing the transition to the proliferative and remodeling phases. In agreement with prior reports, the inventors also observed a delayed healing pattern if ARBs were used throughout all phases of wound healing. This is also is supported by the inventors' prior reports of significant reduction in both PCNA and phospho-Histone H3 in healing skin of the AT₁R^{-/-} mice. In contrast, starting the selective blockade of AT₁R with ARBs and most specifically with 1% valsartan in diabetic and aged mice, as the healing wounds were transitioning to the proliferative phase caused a significant increase in wound blood flow, collagen deposition along with an accelerated rate of healing.

The results of accelerated healing with the use of ARBs (in diabetic and aged mice), contrasted with delayed healing in AT₂R^{-/-} treated with Valsartan may suggest a phase-dependent role for increased AT₂R signaling during the proliferative phase through alterations in TGF-β signaling and alterations in the extracellular matrix. Consistently, the application of topical captopril, which blocks both AT₁R and AT₂R was associated was delayed wound healing. The negative impact of ARBs on wound healing in AT₂R^{-/-} was unexpected. The absence of AT₂R did not simply abolish the beneficial effects of ARBs but was associated with slowed wound healing. Though the exact etiology for this is not clear, insights from the inventors' previous study showing that by day 8 in AT₂R^{-/-} mice, as the healing wounds were transitioning to the proliferative phase, a significant upregulation of wound AT₁R was observed, which may help explain the negative impact for Valsartan on AT₂R^{-/-} mice. Double blockade of AT₁R and AT₂R may have caused the worsening of wound healing in AT₂R^{-/-} mice treated with Valsartan, and may mirror image the negative effects of captopril.

The pattern of healing (increased buildup of slough and plateau of healing rate) seen in placebo treated aged diabetic pigs' wounds resembles the impaired healing seen in older humans with chronic wounds. A key characteristic of chronic wounds is the failure to progress through wound phases and to get “stuck” in inflammatory phase. Cells from patients with chronic wounds also reveal failure of phosphorylation of the SMAD pathway. SMAD proteins are required for signaling in the TGF-beta superfamily. Each Smad has distinct and non-overlapping roles that differ according to tissue type and disease context. Smad1, Smad2, Smad3 and Smad5 transduce ligand-specific signals, whereas Smad4 is an essential common partner of these ligand-specific SMAD proteins. The results shown herein demonstrate selective phosphorylation of Smad3 and inhibition of SMAD1, 2, 5 and 9 with Valsartan treatment. The association between activation of Valsartan induced Smad3 phosphorylation, upregulated co-Smad4 and accelerated rate of healing aligns with prior reports demonstrating augmented wound healing (increased granulation tissue area, number of capillaries, and re-epithelialization rate) with exogenous administration of Smad3 and TGFβ of the wounds. Furthermore, it has been also shown that Smad3 phosphorylation is associated with increased collagen gene transcription and promotes collagen production, which is consistent with the mice and pigs data on increased collagen deposition with topical Valsartan treatment. This increased collagen deposition and improved collagen arrangement provides an important scaffold for healing cells and explains the increased tensile strength of treated skin. The effects of Valsartan on wound collagen deposition and arrangement may open a new vista for the use of topical ARBs in skin wrinkling and in facio-maxillary reconstructive surgery.

Similarly, the knockdown of Smad4 was associated with aberrant wound healing. Wounds from Smad4 knockout mice had higher cell infiltrates and increased degradation adjacent to the migrating epidermal tongue.

Mitochondria provide energy and produce reactive oxygen species to drive the increased mitotic and synthetic activity necessary for wound healing. Several groups demonstrated a link between age-related mitochondrial dysfunction and impaired wound healing. Benigni et al reported an increase in mitochondrial biogenesis in AT₁R knockout mice that was mediated through upregulation of the pro-survival genes nicotinamide phosphoribosyl transferase and sirtuin 3. The identification of a functional intra-mitochondrial angiotensin system (MAS) may provide additional insight into the RAS interface with wound healing. Activation of the intra-mitochondrial AT₂R is coupled to modulation of mitochondrial energy production. The use of topical Valsartan was associated with increase in mitochondrial COX IV, the terminal enzyme complex in the respiratory chain, catalyzing the reduction of molecular oxygen to water coupled to the translocation of protons across the mitochondrial inner membrane to drive ATP synthesis.

As will be shown by the following examples, the 1% valsartan composition of the invention enhances chronic wound healing in diabetic mice and in aging diabetic pigs. The accelerated healing rate was associated with increased wound blood flow, collagen deposition and re-epithelization and led to increased tensile strength of healing skin. The improved skin parameters were associated with selective activation of Smad3 and co-Smad4 along with increased MAPK, α-SMA, VEGF Receptor 2 and higher mitochondrial content in tissues taken from the wound bed.

Having now described the present invention in detail, the same will be more clearly understood by reference to the following examples, which are included herewith for purposes of illustration only and are not intended to be limiting of the invention

### EXAMPLES

### Example 1

### Protocol for Manufacturing a 1% Valsartan Gel Composition

Active Agent: Diovan (valsartan) 320 mg tablets (NDC: 0078-0360-34)

Source: Novartis Pharmaceutical Corp, East Hanover, N.J., USA.

Diluent: 1) Sterile water for injection, 50 mL vials

Source: Hospira, Lake Forest, Ill., USA

2) Surgilube gel (item no.: 0281-0205-37)

Source: Savage Laboratories, Melville, N.Y., USA.

### Supplies for Preparation:

1) 60 mL BD Luer-Lok Tip sterile syringe with BD PrecisionGlide Needle

Source: Becton Dickinson, Franklin Lakes, N.J. 07417

2) PrecisionGlide Sterile Needle, 20G 1½ inch

Source: Becton Dickinson, Franklin Lakes, N.J. 07417

3) 20 dram vials (Friendly and Safe vials)

Source: Health Care Logistics, 50 Town Street, Circleville, Ohio 43113

4) Sterile water for injection, 50 mL vials

Source: Hospira, Lake Forest, Ill., USA

5) Clean Room Wiper, Model 8025

Source: Liberty Industries, 133 Commerce Street, East Berlin, Conn. 06023

6) Glass Beaker

### Storage and Dispensing Container:

Push-Up Ointment Container-140 mL

Source: Health Care Logistics, 50 Town Street, Circleville, Ohio 43113

Procedures for Compounding: Valsartan 1% gel is compounded by mixing valsartan powder with sterile water for injection and Surgilube to make a uniform substance. Valsartan powder is prepared from commercially available Diovan 320 mg tablets. Compounders are required to thoroughly wash and dry their hands and work space. Gloves are worn during all stages of compounding. Compounding surfaces, mortar and pestle, glass beaker, and 20 dram vials are disinfected with Sterile 70% Isopropanol and air dried prior to initiation of any compounding procedures.

### Valsartan Powder Compounding Procedure

1) A compounding Lot number is assigned and a compounding label is prepared.
2) Lot numbers and expiration dates of Valsartan 320 mg tablets, sterile water for injection, and Surgilube are captured on the compound form.
3) An appropriate number of valsartan 320 mg tablets are counted. The count is double-checked.
4) Sterile water for injection is poured on the dry clean room wiper with a sterile syringe to make it wet.
5) Each coated tablet is wiped with a wet clean room wiper until coating is no longer visible.
6) Tablets with removed coating are weighed using the analytical balance and weight is recorded on the compounding form.
7) The ratio of valsartan plus inactive ingredients to valsartan without inactive ingredients is calculated using the following formula: actual weight of tablets without coating (g)÷(# of tablets weighed*0.32 g/tablet).
8) A mortar and pestle is used to crush valsartan tablets.
9) Powder is transferred to the 20 dram vial
10) The vial is capped and labeled with a compounding label.
11) A copy of the compounding label is affixed to the compound form.

### Valsartan 1% Gel Compounding Procedures

1) Amount (g) of valsartan powder+inactive ingredients is calculated using the following formula: (final volume of 1% gel×ratio calculated in step 7)÷100
2) The calculated amount of powder containing valsartan+inactive ingredients is weighed using an analytical balance. The actual weight of the powder is recorded on the compounding form.
3) The weighed valsartan powder is placed into a glass beaker.
4) Sterile water for Injection USP is measured with a sterile 60 mL syringe (3% v/v)
5) Water is slowly added to the powder while mixing with a spatula.
6) Surgilube is added slowly in small quantities mixing well between additions of gel.
7) Once powder particles are completely dissolved and uniform throughout Surgilube, a sufficient volume of Surgilube is added to a final volume.
8) The gel is thoroughly mixed for about 5 minutes.
9) Gel is transferred with a new 60 mL syringe into Push-Up Ointment Containers
10) Each container is labeled and a label is affixed to the compounding form.

### Procedures for Dispensing

### Dispensing of the 1% Valsartan Gel Composition (Treatment B)

1) The specified amount of study drug to be dispensed will be measured and placed into Push-Up Ointment Containers.

### Storage and Stability Conditions:

| | | | | Storage/Stability |
|---|---|---|---|---|
| | | | | after |
| | | | | Recon/Dilution |
| | Strength/ | | Storage/Stability | or change |
| | Dosage | | Before | in Storage |
| Component Name | Form | | Recon/Dilution | Temperature |
| Diovan (Valsartan) | 320 | mg | Room temperature | N/A |
| | tablets | | Store at 25° C. | |
| | | | (77° F.); | |
| | | | Excursions | |
| | | | permitted to | |
| | | | 15-30° C. | |
| | | | (59-86° F.) | |
| Surgilube (Surgical | Gel (density | | Room temperature | N/A |
| Lubricant) | (~1.1 g/mL) | | | |
| Avicel | Powder | | Room temperature | |
| Microcrystalline | | | | |
| Cellulose | | | | |
| Sterile water for | 10 | mL | 20-25° C. | Sterile water |

| | | | |
|---|---|---|---|
| injection | | | for injection |
| Valsartan | 1% gel | Refrigerate 2-8° C. | 60 days |
| Placebo for Valsartan | gel | Refrigerate 2-8° C. | 60 days |

### Example 2

### Protocol for Manufacturing a Placebo Gel Composition (Treatment A and Treatment C)

Agents: 1) Avicel Microcrystalline Cellulose, NF, PH. Eur. JP

Source: FMC BioPolymer, Philadelphia, Pa., USA

2) Surgilube gel

Source: Savage Laboratories, Melville, N.Y., USA

### Supplies for Preparation:

1) 60 mL BD Luer-Lok Tip sterile syringe with BD PrecisionGlide Needle

Source: Becton Dickinson, Franklin Lakes, N.J. 07417.

2) 20 dram vials (Friendly and Safe vials)

Source: Health Care Logistics, 50 Town Street, Circleville, Ohio 43113

### Storage and Dispensing Container

1) Push-Up Ointment Container—140 mL

Source: Health Care Logistics, 50 Town Street, Circleville, Ohio 43113

### Placebo Gel Compounding Procedure

1) An appropriate amount of Avicel Microcrystalline Cellulose powder (2 grams of microcrystalline cellulose per 100 mL of final volume of the gel) is weighed out in a 20 dram vial with an analytical balance with exact weight to be recorded on the compounding form.
2) Avicel Microcrystalline Cellulose powder is placed into a glass beaker
3) Surgilube is added slowly in small quantities mixing well between additions of gel.
4) Once powder particles are completely dissolved and uniform throughout Surgilube, a sufficient volume of Surgilube is added to a final volume.
5) The gel is thoroughly mixed.
6) Gel is transferred with a new 60 mL syringe into Push-Up Ointment Containers 18) Each container is labeled and a label is affixed to the compounding form.

### Procedures for Dispensing

### Dispensing of Placebo Gel Composition

1) The specified amount of placebo gel to be dispensed is measured and placed into Push-Up Ointment Containers.

### Example 3

### Pharmacokinetics of Topical Valsartan in Porcine Model

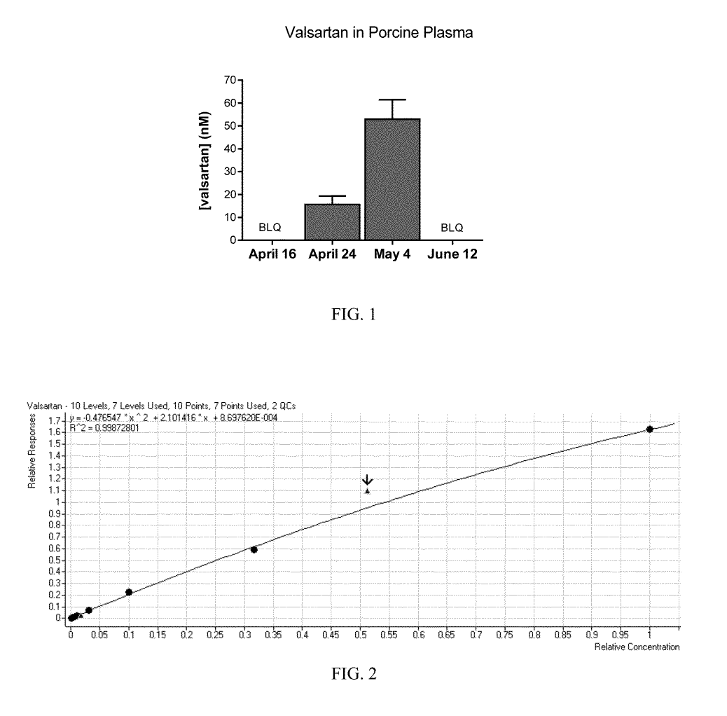

Plasma levels from pigs treated with the valsartan were drawn to determine the potential toxicity. Wounded pigs were treated with topical valsartan. Plasma was collected and stored frozen until analysis for valsartan. The results revealed valsartan plasma concentration ranged from a mean of about 50 nM on May 4 to less than 1 nM (below the limit of quantitation) on June 12. See FIG. 1. Baseline samples (April 16 and June 12) were all below the limit of quantitation (BLQ) and were assigned a value of 0 for graphing.
Analysis Method: Untreated pig plasma was spiked with valsartan at 100 μM through 1 nM at half-log dilutions along with a plasma blank. Plasma standards and samples (50 μL) were extracted in 500 μL methanol containing 100 nM losartan (internal standard). Extracts were centrifuged at 16000×g for 5 minutes at 4° C. to precipitate proteins. Extracts (500 μL) were transferred to a new tube and dried in vacuum at 45° C. for 90 minutes. Samples were reconstituted in 30% acetonitrile in water (50 μL) and centrifuged as above. Supernatants (45 μL) were transferred to a 96 well plate. Analytes (10 μL) were separated on an Agilent 1290 UPLC system with a c18 column using a gradient run of 50-95% acetonitrile over 2 minutes at 0.4 mL/minute and detected on an Agilent 6520 QTOF mass spectrometer. Standards within the quantifiable range were used to generate a standard curve. See FIG. 2 below. The limit of quantitation was 1 nM in porcine plasma.

The plasma concentration found in pigs after topical administration of 1% valsartan gel was 75 lower that the plasma concentrations found in humans after oral administration of valsartan [Saydam, Siddiqui].

### Example 4

### Treatment Plan Outline for Treating Wounded Pig with Valsartan Formulation

Animals: 3 alloxan-induced diabetic Yucatan mini-pigs males, 1 year old.
Medicaments: 1% Valsartan Gel Composition, and Placebo Gel Composition
Wounds: 5 cm diameter rounded full-thickness wounds, applied to the dorsum of the pig (4 wounds per side). Each pig is its own control.
Day 0: wounding
Day 1-7: all wounds receive 10 mL placebo gel (Treatment A) on a daily basis
Day 8-wound closure: each pig receives 10 mL gel B (1% Valsartan gel-Treatment B) on the left side and 10 mL gel C (placebo gel-Treatment C) on the right side. Both gels are applied daily.
Wound measurements: On a daily basis, planimetry and digital photography are conducted to assess changes in wound size.
Upon complete closure: Healed wound tissue is collected and analyzed.

Example 5

### Treatment of Wounded Pigs with 1% Valsartan Formulation

### Summary

Eight 5 cm full thickness circular wounds were successfully created along the dorsal paraspinous areas (4 wounds per side) for each of three diabetic Yucatan miniature swine. After 6 days of daily application of Treatment A to all wounds, followed by 51 days of topical administration of either Treatment B or Treatment C (4 wounds per treatment B or treatment C per animal), there were clear signs of improved wound healing for Treatment B wounds as compared to those receiving Treatment C. The surgical procedures and treatment regimens were well tolerated by all animals.

### Experimental Study Design

The study had two treatment groups, which consisted of a test article treatment or a vehicle control treatment on three diabetic (alloxan-induced) Yucatan miniature swine. The acclimation period was 7 days. On Day 0, each study animal underwent surgery to create 8 (4 per side) 5 cm full-thickness circular excisional wounds on their dorsal surface. All wounds were initially topically treated with the same (Treatment A) compound once daily for the first 7 days (Day 0 through Day 6). Thereafter, 4 wounds per animal received daily topical administration of Treatment B, and the other 4 wounds received Treatment C for the duration of the study. All wound sites (8 per animal) were covered with one dressing type. All wounds were observed/evaluated up through final sample collection (Day 57). The experimental study design, including treatment groups and wound sites/time points, and variables to be evaluated and intervals are presented in Tables 1 through 3.

TABLE 1
Details of Treatment Groups

| Group ID | Treatment Applied |
|---|---|
| 1 | A (Days 0-6); B (Days 7-termination) |
| 2 | A (Days 0-6); C (Days 7-termination) |

TABLE 2
Details of Wound Site Treatments (Beginning on Day 7)

| Animal ID: 7-065 | | Animal ID: 8-023 | | Animal ID: 8-047 | |
|---|---|---|---|---|---|
| Left Side | Right Side | Left Side | Right Side | Left Side | Right Side |
| B | C | B | C | B | C |
| B | C | B | C | B | C |
| B | C | B | C | B | C |
| B | C | B | C | B | C |

TABLE 3
Variables Evaluated and Intervals

| Parameters | Intervals |
|---|---|
| Mortality & morbidity observations | Daily |
| Physical examinations | Day −6 during acclimation |
| Body weight | Once during acclimation (Day −1) |
| | and prior to termination (Day 57) |
| Wound dressing changes | Daily (Days 0 through 57) |
| Wound assessment: | Daily digital photography: Day 0 through Day 44, Day 50 |
| Photographs and Scoring | and Day 57. Planimetry: Day 0, then every other day |
| | beginning on Day 3 through Day 44, Day 50, and Day 57 |
| Clinical Pathology: CBC, Serum | CBC and serum chemistry: Day −5 (during acclimation), Day |
| Chemistries, and Glucose | 10 (pre-dose), Day 18 (pre-dose) and Day 57 (prior to |
| Monitoring | termination); glucose was monitored at least once daily to |
| | keep within targeted glucose range |
| Blood Pressure Measurements | Day 7: Prior to treatment dose and 4 to 6 hours after |
| | treatment dose |
| | Day 8: Prior to treatment dose |
| | Day 18: Prior to treatment dose |
| | Day 57: Prior to termination |
| Plasma Collections | Prior to surgery (Day 0), Day 10 (pre-dose), Day 18 |
| | (pre-dose), and Day 57 (prior to termination); |
| Study Termination | Day 57 |

### Materials and Methods

### Test Article Information

Name: Treatment A (Gel A); Batch No./Exp Date: 09D14-10/9 Jun. 2014
Name: Treatment B (Gel B); Batch No./Exp Date: 09D14-4/9 Jun. 2014 and 21E14-2/21 Jul. 2014.
Name: Treatment C (Gel C); Batch No./Exp Date: 09D14-6/9 Jun. 2014 and 21E14-3/21 Jul. 2014

### Test Article Preparation

The test articles were provided as “ready-to-use” for administration.

### Test Article Return

Following completion of the in-life phase of the study, all remaining test article was discarded as agreed by Sponsor and Study Director.
Test System

Animals
Species: Sus scrofa, miniature swine
Strain/Gender: Yucatan (diabetic)/Male
Source: Sinclair Bioresources (Auxvasse, Mo.)
Age: 3 years on Day 0
Weight: 38.6 to 60.8 kg (Day −1)
Number: 3
Identification: Numbered ear tag and cage card

Animal Health

Prior to this study, selected male animals had been castrated and fitted with vascular access ports (VAP) for diabetic induction. After these animals had fully recovered from VAP procedures, diabetes was chemically-induced using alloxan. Following induction, the glucose levels of the animals was monitored and regulated with Vetsulin (or equivalent). A glucometer and/or other appropriate device was used to determine glucose levels of the animals. VAP procedures, diabetic induction, and recovery periods were conducted under a separate SRC standard protocol prior to the initiation of this study. Glucose levels of all study animals were monitored and regulated at least once daily during the study to keep within targeted glucose range (target 200 to 600 mg/dL). Blood pressure was determined for each animal on Days 7, 8, 18, and 57 (prior to termination).

Animal Housing
Cage/Pen Design: Animals were individually-housed in pens, appropriate for the size of the animals. The pens were constructed of stainless steel. Elevated flooring of pens was self-spanned polyvinyl chloride (PVC)-coated expanded metal flooring. No bedding was used in the pens.
Environment: The housing room was set to maintain a room temperature of 16 to 27° C. (61 to 81° F.) with fluorescent lights providing a 12-hour light/12-hour dark photoperiod. Relative humidity in the housing room(s) was recorded.

Physical Examination

On Day -6, all animals were given a physical examination by an SRC veterinarian. Examinations included, but were not limited to, examination of the skin (particularly dose site) and external ears, eyes, abdomen, neurological, behavior, and general body condition. Bilateral cataracts were observed in all animals and all animals were accepted for study inclusion.

Diet and Water

The miniature swine were fed standard SRC swine diet S-9 daily at an appropriate amount. Animals had ad libitum access to deep well water.

Acclimation Period

The animals were acclimated for 7 days prior to the initiation of dosing (Day 0). Observation records were maintained during acclimation.

### Dosing Procedure

Fasting and Pre-Anesthetic

All animals were food-fasted for at least 8 hours prior to Day 0 surgical procedures and any additional study procedures that required anesthesia.

Induction and Maintenance

On Day 0, anesthesia of the animals was induced with a combination of Telazol (˜2.2 mg/kg, IM) and Xylazine (˜0.44 mg/kg, IM). Each animal was intubated endotracheally and maintained using isoflurane (0.5 to 5% in 100% oxygen). On follow-up days, anesthesia of the animals was induced and maintained with direct administration of isoflurane (0.5 to 5% in 100% oxygen) through a nose mask.

Day 0 Surgical Procedure

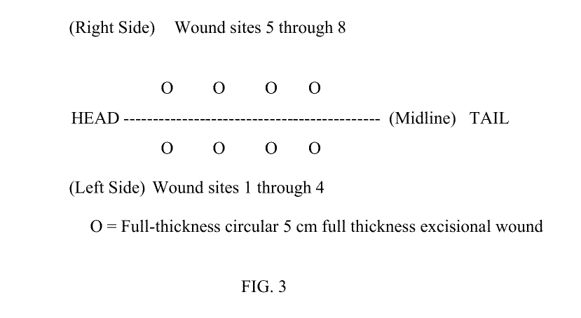

On Day 0, the dorso-lateral back area of each animal was closely clipped with electric clippers. Each animal was prepared for surgery using alternating disinfecting scrubs and isopropyl alcohol rinses. The surgical area was draped and a sufficiently large hole(s) was created within the drape(s). The proposed wound sites were outlined using a sterile template and sterile marker. Each animal had eight 5 cm diameter circular wound sites designated (one row of 4 wounds/side), spaced at least 4 cm apart. Refer to FIG. 3.
a. Using a surgical blade, eight 5 cm full-thickness circular excisional wounds were created (down to the fat layer). All excised tissue from each wound site was discarded.
b. Direct pressure was utilized to obtain hemostasis.
c. Wound assessments, including photograph(s), measurements, and scoring (general observations), were performed for all wounds.
d. Approximately 10 mL of Treatment A was topically administered to each wound and to cover the entire wound using a syringe
e. Each wound site was covered with gauze dressing which was secured in place with tape. In addition, the entire wound area was covered with a compression dressing and/or a tear-resistant mesh (stockinette) to minimize dislodgement of the dressing material.
f. Each animal was monitored until it had completely recovered from the anesthesia.

Follow-Up Procedures

On Days 1 through 56, dosing and wound dressing procedures were performed for each study animal. Minimal general anesthetic was use until they are acclimated to dressing changes. Animals undergoing anesthesia for dosing/wound dressing procedures were fasted overnight prior to anesthesia. If needed, anesthesia was induced and maintained, using isoflurane (0.5 to 5% in 100% oxygen) by an SRC veterinarian and/or qualified technician under the SRC veterinarian's supervision. The following procedures were performed:
a. The occlusive dressings were carefully removed and discarded.
b. The dorso-lateral back area of each animal was closely clipped with electric clippers, if necessary.
c. The dorso-lateral back area of each animal was prepared for fresh wound dressing using isopropyl alcohol soaked-gauze and/or dry gauze.
d. Wound assessments, including photograph(s), measurements, and scoring (general observations), were performed as per Table 3.
e. On Days 0 through 6, all wounds of all animals received topical administration of approximately 10 mL of Treatment A. Beginning on Day 7, the appropriate amount of Treatment B or C (5 to 10 mL) was topically-administered. Refer to Table 1 and Table 2 for treatment assignments.
f. Each wound site was covered with gauze dressing which was secured in place with tape. In addition, the entire wound area was covered with a compression dressing and/or a tear-resistant mesh (stockinette) to minimize dislodgement of the dressing material.
g. Each animal was monitored until it had completely recovered from anesthesia (if employed).

Refinement

All study animals were under general anesthesia for surgery and dose administration procedures. On Days 0 pre surgery, buprenorphine (0.02 mg/kg, IM) was administered to each animal. Additionally tramadol were administered, on Day 0 through 5 as recommended by an SRC veterinarian.

### Daily Mortality/Moribundity

General in-cage/pen observations for mortality/moribundity were made at least daily.

### Body Weights

Animals were weighed once during acclimation (Day −1) and again prior to termination (Day 57).

### Wound Observations

The following observations/evaluations were performed on all wound sites for all study animals according to Table 3.
Wound Site Observations: Prior to each dose application, wounds were generally observed for signs of infection, hemorrhage and/or healing.
Digital Photographs: Daily high resolution photograph procedures were performed. All photographs contained appropriate identifiers (animal ID, site #, date, reference scale) for identification along with a “color” scale. Each photograph was taken from directly in front of the wound in order to ensure an accurate measurement. The wound, ruler, label, and “color” scale filled the frame, with the ruler positioned as flat as possible. Appropriate identifiers were used to orient the wounds from head to tail.
Wound Planimetry: Adobe PhotoShop software was used for planimetric measurement of wound areas from the digital photographs.

### Clinical Pathology

Day −5 (acclimation), Day 10 (pre-dose), Day 18 (pre-dose) and Day 57 (prior to termination); blood samples for CBC and serum chemistries were collected for clinical pathology analysis from each animal and analyzed by SRC.
Hematology: Blood samples (3 mL/animal) were collected from all animals. K₃EDTA was used as anticoagulant. Hematology samples were stored refrigerated or with ice packs until analysis. The hematology analysis included but was not necessarily limited to:

| | | |
|---|---|---|
| White Blood Cell Count | Mean Cell Hemoglobin Conc. | Monocytes (% and absolute) |
| Red Blood Cell Count | Differential White Blood Cell Count | Platelet Count |
| Hemoglobin | Neutrophils (% and absolute) | |
| Hematocrit | Eosinophils (% and absolute) | |
| Mean Cell Volume | Basophils (% and absolute) | |
| Mean Cell Hemoglobin | Lymphocytes (% and absolute) | |

Serum Chemistries: Blood samples (5 mL/animal) were collected from all animals food fasted overnight. Serum was prepared by centrifuging for approximately 15 minutes at 3000 rpm at 4° C. Serum samples were stored in a refrigerator or with ice packs until analysis. The serum chemistry included but was not necessarily limited to:

| | | |
|---|---|---|
| Alanine aminotransferase (ALT) | Calcium | Inorganic Phosphorus |
| Albumin | Chloride | Potassium |
| Albumin/ Globulin Ratio | Cholesterol | Sodium |
| Alkaline Phosphatase (ALP) | Creatinine | Total Bilirubin |
| Aspartate Aminotransferase (AST) | Globulin | Total Protein |
| Blood Urea Nitrogen (BUN) | Glucose | Triglycerides |

### Plasma Collections

Prior to surgery (Day 0), Day 10 (pre-dose), Day 18 (pre-dose), and Day 57 (prior to termination); blood samples (3 mL/animal) were collected, processed to plasma by centrifuging for approximately 15 minutes at 3000 rpm at 4° C., transferred into labeled cryovials, immediately frozen on dry ice, and shipped to the sponsor for future analysis.

### Anatomic Pathology

Macroscopic Examination

All animals were humanely euthanized at the end of the study. No gross anatomic assessment was performed.

Wound Tensiometry

At termination, an approximately 8 mm×16 mm tensiometry sample was collected from the dorsal side of each wound site that spanned the cranial to caudal aspect of the wound for analysis. This procedure was performed and data maintained by sponsor personnel. No details of analysis or results were included in this in-life report.

Microscopic Examination

An approximately 3 cm×5 cm histology sample was collected from the ventral side of each wound site that spanned the cranial to caudal aspect of the wound including healthy tissue outside the wound site and placed in 10% NBF for possible analysis. These collected tissue samples were shipped to the sponsor. No details of analysis or results were included in this in-life report.

### Results

Wound Creation and Dosing

All animals tolerated anesthesia and surgical procedures well. All wounds were successfully created without complication. Designated treatments were applied to each wound prior to dressing procedures. For the first 30 days of the study, each wound was treated with approximately 10 mL of designated test article. Subsequently, dosing volumes were adjusted downward to 5 mL as wound areas decreased during the remainder of the study.

Daily Mortality/Moribundity

There was no mortality and no instances of individual animal moribundity over the course of the study.

Animal Health

All animals remained healthy for the duration of the study, without signs of adverse reactions to the treatment regimen. Daily blood glucose monitoring results showed expected variation over the course of the study, and generally remained within the target range for diabetic swine. Blood pressure results showed some variation between animals, but no patterns or trends over time suggesting an adverse reaction to test article administration. Refer to Tables 4-6 for individual animal data.

TABLE 4
Individual Animal Information, Body Weight and Blood
Glucose Summary

| | | | Body | Body | |
|---|---|---|---|---|---|
| | | Age at | Weight | Weight | Blood Glucose |
| Animal | Date of | Day 0 | (kg) | (kg) | Avg. (Range) |
| ID | Birth | (years) | Day (−1) | Day 57 | (mg/dL) |
| 7-065 | May 7, 2011 | 3 | 52.3 | 52.4 | 404 |
| | | | | | (121-666) |
| 8-023 | Jun. 8, 2011 | 3 | 38.6 | 43.8 | 474 |
| | | | | | (245-724) |
| 8-047 | Jun. 13, 2011 | 3 | 60.8 | 61.1 | 386 |
| | | | | | (71-573) |

TABLE 5
Individual Animal Blood Glucose Levels (mg/dL)

| | Animal ID | | | | | |
|---|---|---|---|---|---|---|
| | 7-065 | | 8-023 | | 8-047 | |
| | Time Point | | | | | |
| | AM | PM | AM | PM | AM | PM |
| Day (−6) | 433 | 163 | 382 | 213 | 343 | 231 |
| Day (−5) | — | 397 | — | 207 | — | 378 |
| Day (−4) | 383 | 277 | 247 | 201 | 514 | 489 |
| Day (−3) | 363 | 309 | 374 | 397 | 379 | 219 |
| Day (−2) | 317 | — | 314 | — | 276 | — |
| Day (−1) | 441 | — | 528 | — | 644 | — |
| Day 0 | — | 292 | — | 351 | — | 323 |
| Day 1 | 439 | 468 | 464 | 496 | 408 | 539 |
| Day 2 | 249 | 501 | 486 | 377 | 450 | 531 |
| Day 3 | 461 | 442 | 314 | 377 | 471 | 226 |
| Day 4 | 469 | 412 | 368 | 492 | 444 | 406 |
| Day 5 | 466 | 309 | 558 | 405 | 479 | 354 |
| Day 6 | 464 | 378 | 449 | 406 | 488 | 421 |
| Day 7 | 526 | 364 | 543 | 304 | 432 | 378 |
| Day 8 | 524 | 379 | 592 | 420 | 448 | 361 |
| Day 9 | 648 | 447 | 578 | 420 | 471 | 450 |
| Day 10 | 666 | 397 | 474 | 398 | 419 | 485 |
| Day 11 | 548 | 387 | 627 | 411 | 388 | 361 |
| Day 12 | 554 | 498 | 523 | 411 | 475 | 426 |
| Day 13 | 511 | 197 | 542 | 428 | 492 | 437 |
| Day 14 | 477 | 397 | 598 | 448 | 469 | 307 |
| Day 15 | 499 | 478 | 546 | 590 | 450 | 364 |
| Day 16 | 321 | 483 | 667 | 574 | 448 | 461 |
| Day 17 | 431 | 391 | 488 | 457 | 413 | 253 |
| Day 18 | 399 | 429 | 650 | 562 | 416 | 313 |
| Day 19 | 514 | 362 | 562 | 428 | 407 | 192 |
| Day 20 | 533 | 429 | 564 | 674 | 390 | 301 |
| Day 21 | 545 | 360 | 534 | 489 | 462 | 234 |
| Day 22 | 487 | 273 | 508 | 531 | 475 | 456 |
| Day 23 | 540 | 246 | 513 | 401 | 536 | 327 |
| Day 24 | 471 | 406 | 624 | 534 | 421 | 336 |
| Day 25 | 511 | 426 | 605 | 483 | 436 | 351 |
| Day 26 | 615 | 401 | 654 | 527 | 490 | 283 |
| Day 27 | 487 | 427 | 619 | 523 | 518 | 419 |
| Day 28 | 488 | 361 | 489 | 568 | 463 | 296 |
| Day 29 | 476 | 495 | 538 | 596 | 556 | 370 |
| Day 30 | 376 | 325 | 510 | 523 | 394 | 359 |
| Day 31 | 382 | 364 | 486 | 724 | 357 | 447 |
| Day 32 | 505 | 315 | 521 | 436 | 449 | 559 |
| Day 33 | 470 | 286 | 480 | 607 | 420 | 76 |
| Day 34 | 461 | 365 | 429 | 314 | 392 | 422 |
| Day 35 | 398 | — | 440 | — | 427 | — |
| Day 36 | — | 293 | — | 421 | — | 309 |
| Day 37 | 325 | 347 | 425 | 513 | 317 | 353 |
| Day 38 | 508 | 465 | 411 | 584 | 443 | 376 |
| Day 39 | 495 | 299 | 380 | 419 | 434 | 185 |
| Day 40 | 557 | 384 | 423 | 489 | 465 | 352 |
| Day 41 | 613 | 365 | 518 | 511 | 573 | 87 |
| Day 42 | 396 | 232 | 496 | 422 | 476 | 250 |
| Day 43 | 436 | 270 | 475 | 482 | 464 | 274 |
| Day 44 | 453 | 185 | 425 | 427 | 309 | 400 |
| Day 45 | 415 | 410 | 428 | 497 | 306 | 411 |
| Day 46 | 526 | 364 | 510 | 466 | 520 | 537 |
| Day 47 | 478 | 393 | 372 | 357 | 385 | 338 |
| Day 48 | 362 | 361 | 389 | 412 | 401 | 236 |
| Day 49 | — | 282 | — | 435 | — | 405 |
| Day 50 | — | 272 | — | 377 | — | 296 |
| Day 51 | 362 | 161 | 428 | 360 | 402 | 163 |
| Day 52 | 447 | 234 | 469 | 390 | 429 | 295 |
| Day 53 | 329 | 256 | 403 | 315 | 286 | 185 |
| Day 54 | 309 | 226 | 253 | 373 | 400 | 220 |
| Day 55 | 242 | 210 | 459 | 426 | 489 | 399 |
| Day 56 | 405 | 123 | 399 | 297 | 412 | 71 |
| Day 57 | 461 | 121 | 341 | 245 | 355 | 304 |

TABLE 6
Individual Animal Blood Pressures

| Animal | Day 7 | Day 7 | Day 8 | Day 18 | Term |
|---|---|---|---|---|---|
| ID | Pre-Dose | Post-Dose | Pre-Dose | Pre-Dose | Pre-Dose |
| 7-065 | 173/130 | 181/143 | 112/66 | 196/117 | 154/124/140 |
| 8-023 | 140/99 | 154/131 | 163/59 | 173/80 | 167/106/149 |
| 8-047 | 151/106 | 167/126 | 88/41 | 133/105 | 138/103/121 |

Blood pressure expressed as systolic/diastolic or systolic/diastolic/mean

### Body Weights

All animals maintained/gained body weight over the course of the study (Table 4).

### Wound Observation

General Appearance

Wounds generally showed signs of normal healing, with occasional mucopurulent discharge noted for some wounds. There were no instances of obvious infection or hemorrhage.

Digital Photographs

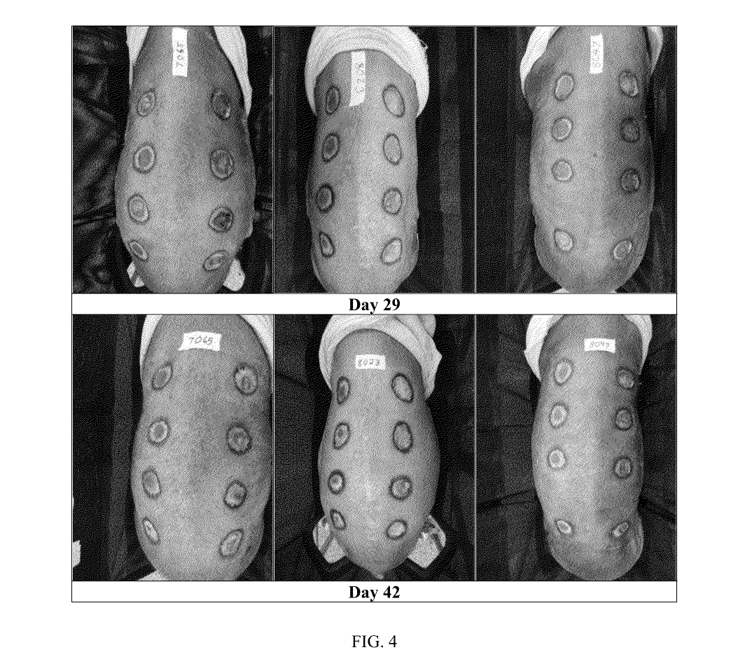

Although there was some animal to animal variability digital photographic images consistently demonstrated clear improvement of healing rates for wounds treated with Treatment B as compared to those administered Treatment C for all three animals. Representative photographs are shown in FIG. 4.

Wound Planimetry

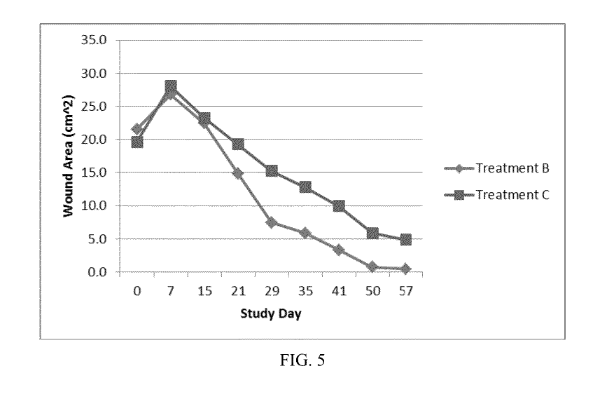

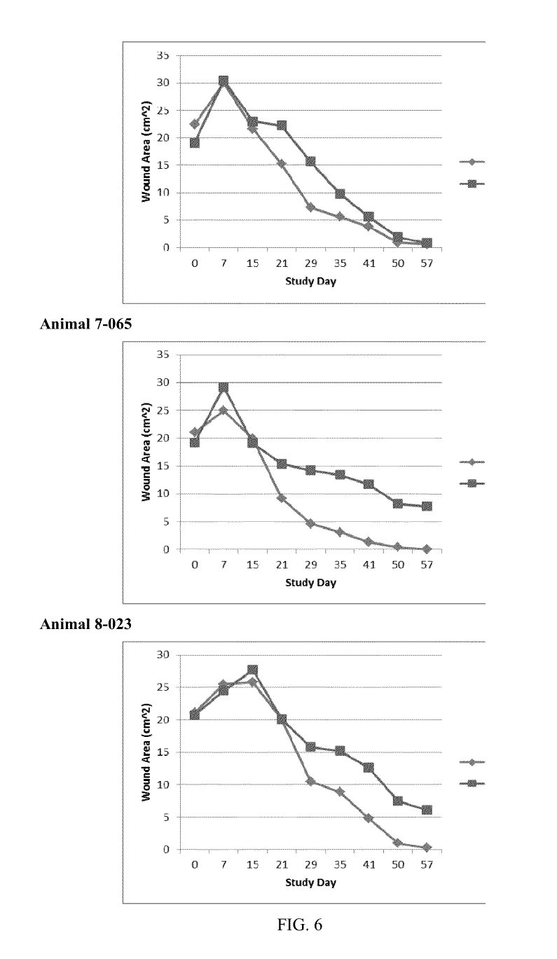

Consistent with the photographic results, wounds receiving Treatment B generally showed faster healing rates as compared to corresponding Treatment C wounds. The healing rates by wound treatment for combined animal results, and for each individual animal are provided in FIG. 5 and FIG. 6, respectively.

### Clinical Pathology

Hematology and serum chemistry analyses were performed on blood samples obtained prior to dosing, on Day 18, and again just prior to study termination (Day 57). These results are provided in Tables 4-6. There was an apparent trend for absolute and relative neutrophil counts to decrease in all three animals between pre-dose and termination time points. This isolated finding is consistent with normal biologic variation, and not considered to be an adverse effect of test article exposure. There were no significant serum chemistry findings at any time point.

### Conclusions

Three diabetic Yucatan miniature swine successfully underwent surgical creation of eight circular 5 cm diameter (˜20 cm²) full thickness excisional skin wounds on the paraspinous areas (4 per side) under general anesthesia. Each wound was treated with daily topical application of approximately 10 mL of Treatment A for 7 days. Subsequently, 4 wounds per side of each animal were treated daily with approximately 10 mL of either Treatment B (left side) or Treatment C (right side) for the duration of the study. All animals tolerated study procedures well. There was no observed adverse animal health effects found based on monitoring of body weights, blood pressure, blood glucose levels, or clinical pathology testing (serum chemistry and hematology).

Over the course of the study, there was a clear trend for wounds exposed to Treatment B (1% valsartan) to show accelerated healing rates as compared to those receiving Treatment C (placebo).

### Example 6

### Pathology Assessment

In the pig model described in Example 5, evaluation of the wounds treated by Treatment B (1% valsartan; Group 1) and Treatment C (placebo; Group 2) was completed via the following five criteria:
1) Wound closure: Proportion of the samples showing a closed wound on the path slide.
2) Wound gap: Wound gap is the distance between the epithelial margins of the wound. It was measured only when wounds were NOT closed.
3) Epithelial grade: This is a grading system derived from a previous publication [Marti, et al.]. Quality of epithelial healing is graded according to the number of cell layers and maturity:

| Grade | description |
|---|---|
| 0 | Wound not closed |
| 1 | Epithelium displays 1 to 3 layers of cells |
| 2 | Epithelium displays 4 or more layers of |
| | cells |
| 3 | Epithelium is mature and well reticulated |

4) Epithelial thickness: The method for calculating the epithelial and dermal thickness is derived from a previous publication [Dou, et al.] Starting from the center of the wound, 3 measures of the epithelial thickness were taken, the center plus 2 measures at equidistance from it, utilizing Motic* software, specific to the microscope used to capture the images. The number associated with each sample was the average of these three measurements.
5) Dermal thickness: The same method of measurement was used. Three equidistant measures starting from the center.

### Results

1) Wound closure:

| | Group 1 | Group 2 |
|---|---|---|
| Wounds closed | 100% | 50% |
| Wounds open | 0 | 50% |
| n | 11 | 8 |

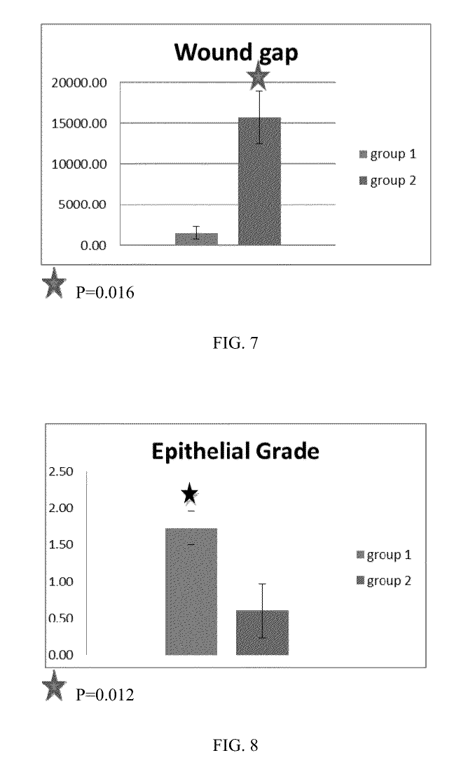

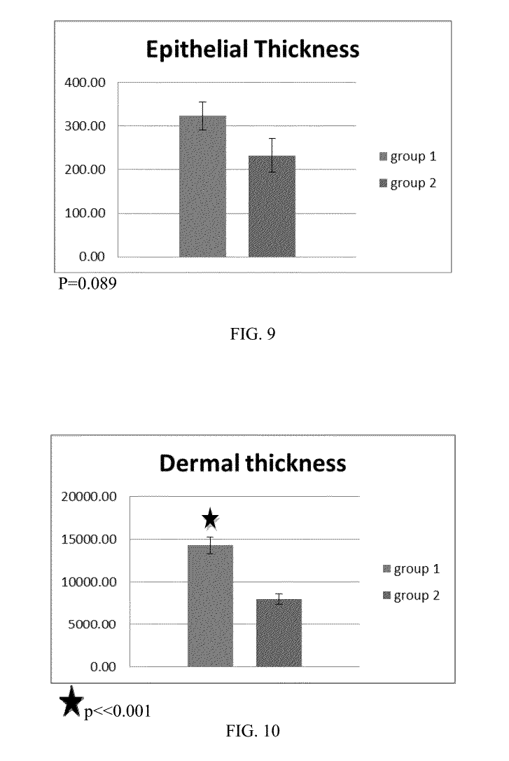

All the wounds are microscopically closed in group 1. Only half of the group 2 are.
2) Wound gap: These measurements were taken from slides showing absence of epithelial closure. Results are shown in FIG. 7.
3) Epithelial grade: Results are shown in FIG. 8.
4) Epithelial thickness: Results are shown in FIG. 9.
5) Dermal thickness: Results are shown in FIG. 10.

### Raw Data:

| Group 1 | closed | wound | epith | dermal | epith |
|---|---|---|---|---|---|
| (Treatment B) | W | gap | thickness | thickness | grade |
| A1 | 1 | 0 | 269 | 11980 | 2 |
| A2 | 1 | 5700 | 437 | 14000 | 2 |
| A3 | | 0 | 212 | 12840 | 0 |
| B1 | 1 | 0 | 304 | 13557 | 1 |
| B2 | | 0 | 461 | 19297 | 3 |
| B3 | 1 | 3470 | 217 | 12477 | 1 |
| C1 | 1 | 6570 | 284 | 9250 | 2 |
| C2 | 1 | 0 | 394 | 19993 | 3 |
| D1 | 1 | 0 | 150 | 15293 | 1 |
| D2 | 1 | 0 | 392 | 12027 | 2 |
| D3 | 1 | 1040 | 438 | 16617 | 2 |
| AVE | 1 | 1525.45 | 323.34 | 14302.73 | 1.73 |
| STDEV | 0 | 2513.57 | 106.38 | 3254.01 | 0.90 |
| n | 11 | 11 | 11 | 11 | 11 |
| SQRTn | 3.32 | 3.32 | 3.32 | 3.32 | 3.32 |
| STDERR | 0 | 757.87 | 32.08 | 981.12 | 0.27 |

| Group 2 | closed | wound | epith | dermal | epith |
|---|---|---|---|---|---|
| (Treatment C) | W | gap | thickness | thickness | grade |
| E2 | 1 | 2230 | 535 | 11840 | 2 |
| E3 | 0 | 22470 | 107 | 5617 | 0 |
| F1 | 1 | 14500 | 230 | 8543 | 2 |
| F2 | 0 | 23780 | 226 | 9347 | 0 |
| G1 | 0 | 30620 | 100 | 6660 | 0 |
| G2 | 0 | 22910 | 290 | 7125 | 0 |
| G3 | 1 | 5560 | 263 | 8673 | 0 |
| H1 | | 19550 | 130 | 6350 | 0 |
| H2 | | 15468 | 255 | 6461 | 0 |
| H3 | 1 | 0 | 190 | 9657 | 2 |
| AVE | 0.50 | 15708.80 | 232.63 | 8027.23 | 0.60 |
| STDEV | 0.53 | 10180.46 | 125.72 | 1925.80 | 0.97 |
| n | 8 | 10 | 10 | 10 | 10 |
| SQRTn | 2.83 | 3.16 | 3.16 | 3.16 | 3.16 |
| STDERR | 0.19 | 3219.34 | 39.76 | 608.99 | 0.31 |

### Example 7

### Valsartan Dosage and Wound Healing

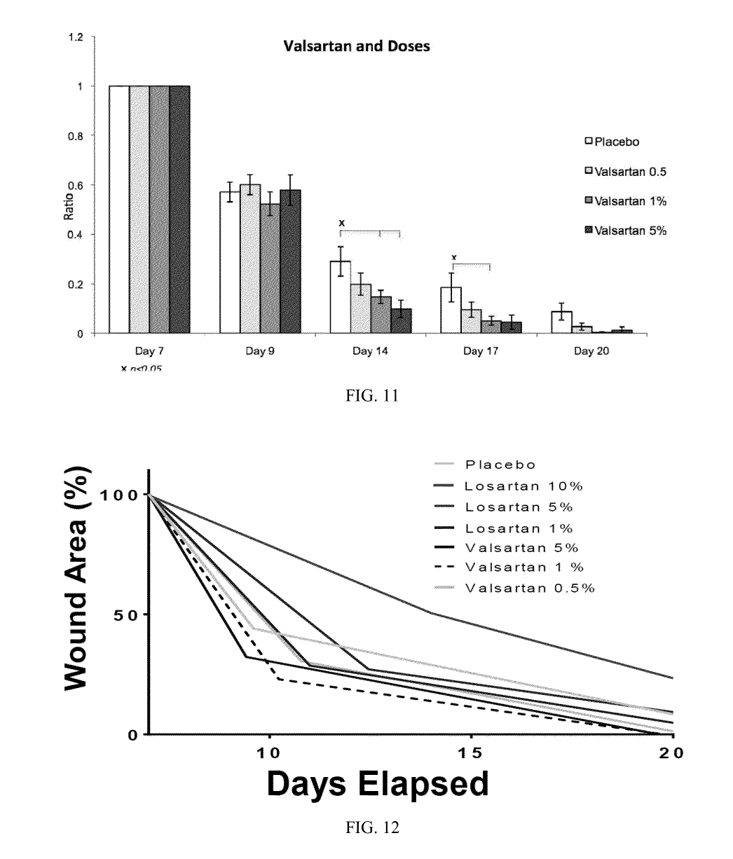

In mouse model wound healing experiments, the dosage of valsartan was varied and the effect of dosage on wound healing was measured. As shown in FIG. 11, valsartan in a dosage of 1% by weight of the topical formulation provided wound healing results that were superior to the results achieved with placebo or with 0.5% or 5% weight valsartan formulations. In FIG. 11, the “ratio” (y-axis) reflects the percentage of wound closure as compared to the wound size on day 7 in the same animal.

### Example 8

### Comparison of Wounds Treated with 1% Valsartan Formulation and Alternative Wound Treatment Therapies

In mouse model wound healing experiments, wound healing was assessed for a number of therapies, including the 1% valsartan formulation of the invention and various conventional wound healing therapies.

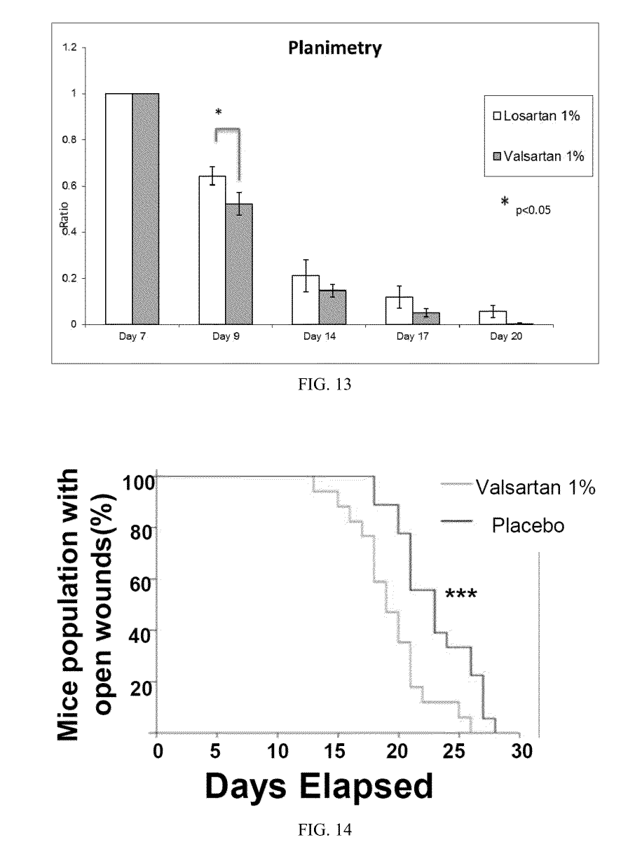

Topical Losartan vs. Valsartan: Despite the similarity in specificity of different ARBs toward AT₁R, each ARB has its unique properties, affinity to AT₁R and impact on cellular functions¹⁶. The efficacy of 1%, 5%, and 10% losartan gel was compared to 0.5%, 1% and 5% Valsartan gel applied during proliferation/remodeling phase of wound healing in diabetic mice. Results (FIG. 12) demonstrated that valsartan was more efficacious in accelerating wound healing compared to losartan. A head-to-head comparison of 1% valsartan and 1% losartan is shown in FIG. 13. Statistically, even though each valsartan dose significantly accelerated healing compared to placebo, there was no significant difference in time of healing between any of the Valsartan doses. However the smallest wound area compared to placebo was seen with Valsartan 1% (P<0.01). Additionally, a Kaplan Meier analysis revealed that when 50% of the animals in the Valsartan 1% cohort achieved complete wound healing, only 10% in placebo treated mice were closed (P<0.001) (FIG. 14). In contrast, application of 10% Losartan was associated with worse wound healing (P<0.05), suggesting possible toxicity related to that higher dose.

To determine the etiology behind the differential effects of best dose of Valsartan 1% as compared to best dose of Losartan 1%, changes in mRNA expression of the angiotensin receptors (AT₁R and AT₂R) and the three isoforms of TGFβ (1, 2 and 3) were examined in wounds treated with Valsartan 1% and Losartan 1%. Wounds treated with 1% Valsartan had lower AT₁R mRNA quantity (0.6 fold, P<0.05) as compared to Losartan 1%. Valsartan treatments also caused statistically non-significant increase in the AT₂R mRNA and a decrease in TGFβ3 mRNA (0.6 fold, P<0.05). These findings provided rationale for the choice of 1% Valsartan as dose for the experiments described below.

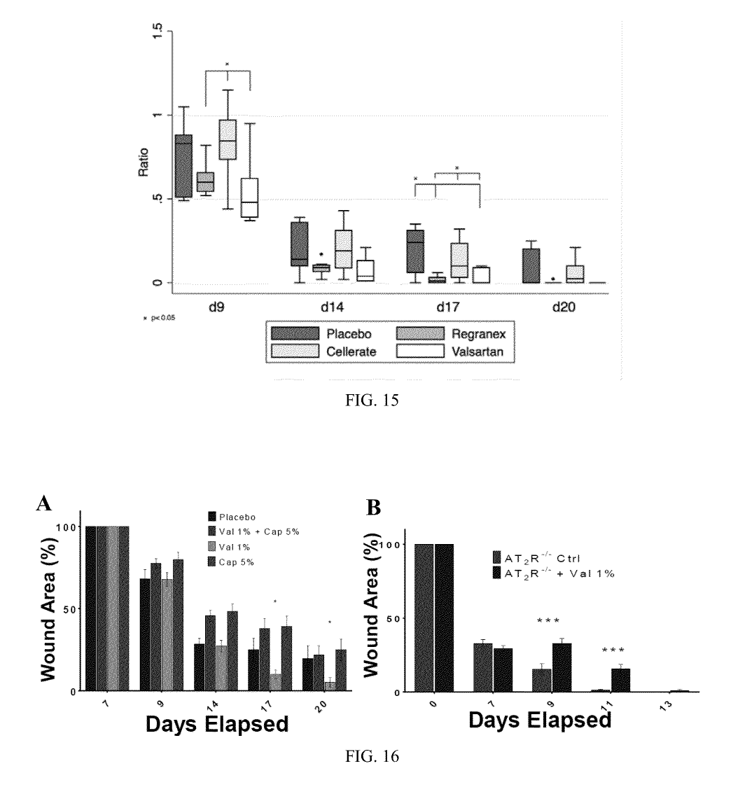

The 1% valsartan formulation of the invention was also compared to wound treatment therapies recognized as currently being best of the market: CellerateRX® (Type I bovine collagen) gel and and Regranex® (becaplermin) gel. FIG. 15 shows planimetric measurements taken over time of a wound treated with one of: placebo, CellerateRX®, Regranex®, or the 1% valsartan formulation of the invention. The 1% valsartan formulation showed improved wound healing properties as compared to CellerateRX®, and was as effective in treating wounds as Regranex®.

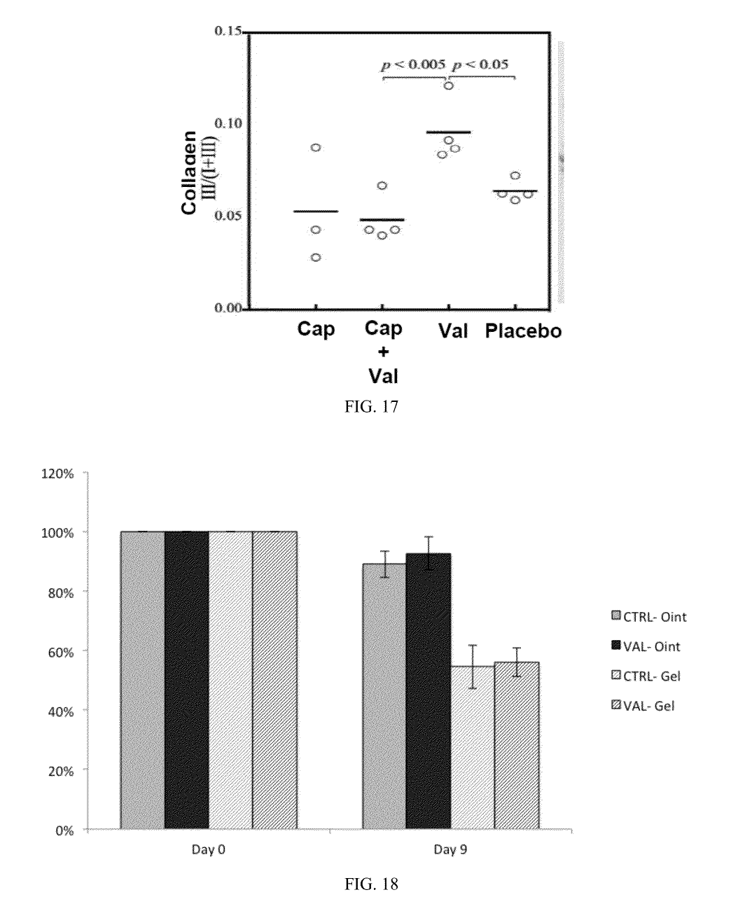

Topical Valsartan vs. Captopril: Clinically, both ARBs and Angiotensin Converting Enzyme (ACE) inhibitors have yielded comparable results in terms of blood pressure control and cardiovascular protection¹⁷. Pharmacologically, ARBs and ACE inhibitors differ on their mechanism of action and the level at which they block RAS. While ARBs block RAS distally at the AT₁R level, ACEi block the conversion of Angiotensin I to Angiotensin II and thereby diminishing available Angiotensin II to bind to either AT₁R or AT₂Rs. These data show that topical treatment with Captopril 5%¹⁸, significantly delayed wound closure rate as compared to Valsartan 1% (P<0.05). Interestingly, the addition of Valsartan 1% to Captopril 5% did not alleviate the negative effects of Captopril (FIG. 16, panel A). Given the mechanistic difference between the agents described above, the contrast between Valsartan and Captopril may suggest a role for AT₂R in mediating the effects of topical Valsartan. To further clarify the possibility of a role for AT₂R in wound healing, Valsartan 1% gel was applied to AT₂R^{-/-} mice. These data (FIG. 16, panel B) suggest that Valsartan 1% paradoxically delayed wound healing in AT₂R^{-/-} mice (P<0.001 at day 9 and day 11). Taken together, this suggests that application of 1% Valsartan gel starting at day 7 after wounding accelerated time to wound closure, and that AT₂R plays a role in mediating the effects of topical Valsartan. This data also suggests that topical treatment with 1% Valsartan also increased the ratio of type III to total collagen as compared to placebo, captopril, or the combination of captopril and valsartan (P<0.05; FIG. 17). Collagen is a marker for assessing skin strength.

Different pharmaceutical carriers were assessed in wound healing experiments. As shown in FIG. 18, the gel formulation of 1% valsartan is superior to an ointment formulation of 1% valsartan in wound healing efficacy.

### Example 9

### Further Testing of the 1% Valsartan Formulation in an Aged and Diabetic Porcine Model of Wound Healing

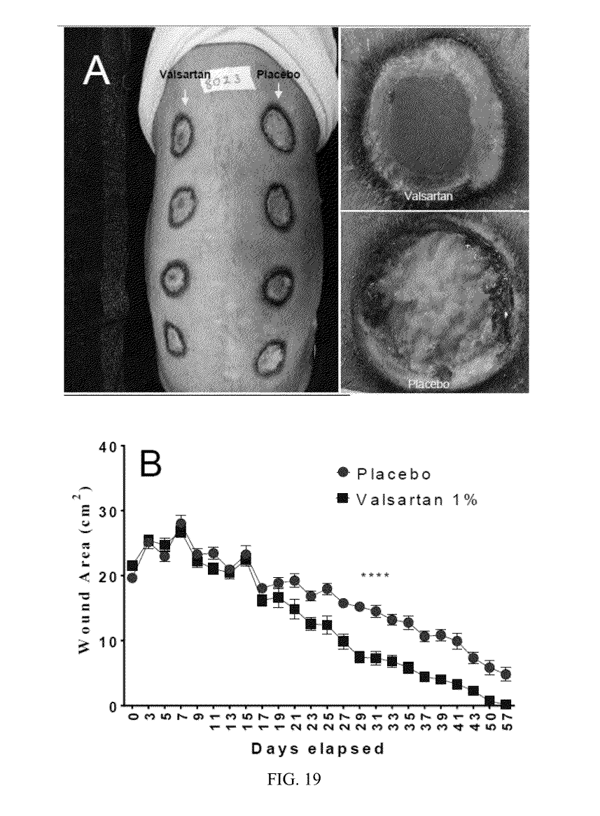

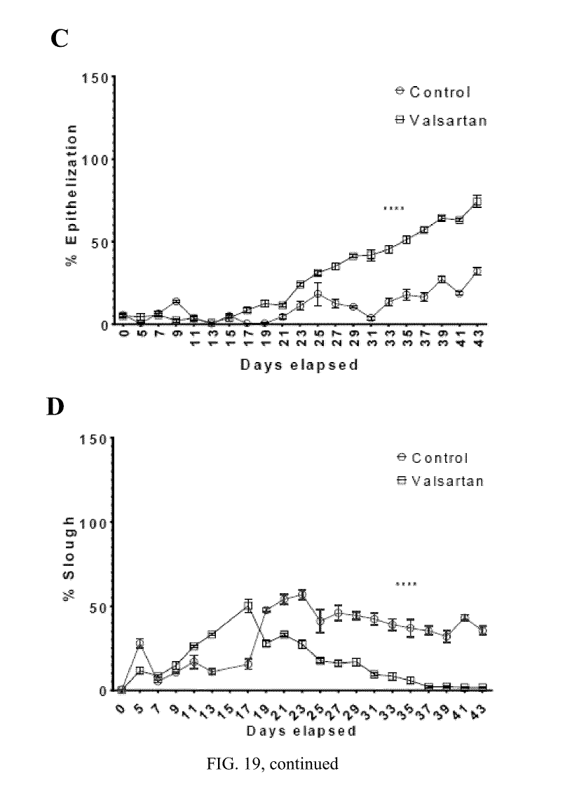

Several of the initially promising products tested for wound healing in mice failed to give positive results when also tried in higher animals or humans. This may in part be due to the differences between rodent skin and humans. Pig skin has been shown to have similar physio-histological properties to human skin and is suggested as a good model for human wounds. Driven by the promising effects of 1% Valsartan gel on accelerating wound healing in mice, the effects of this agent Valsartan gel on aged and diabetic porcine model of chronic wound healing was investigated. Animals were 3 years old at the time of wounding with blood sugars allowed to range between 200-400 gm/dl in order to approximate older diabetic humans with poor glucose control. Wounds treated with Valsartan 1% exhibited superior healing as compared to those treated with placebo gel (FIG. 19, panel A). Consistent with these photographic results, wounds receiving Valsartan showed faster wound closure rates compared to corresponding placebo gel treated wounds over a period of 57 days (FIG. 19, panel B; P<0.0001). All wounds treated with 1% Valsartan gel were closed at day 50 as compared to none of the placebo treated wounds. Using automated digital analysis of daily wound images to monitor changes in different wound compartments, higher rates of epithelization (P<0.0001; FIG. 19, panel C) and lower accumulation of slough at the wound base (P<0.0001; FIG. 19, panel D) was demonstrated in Valsartan treated wounds.

### Example 10

### Selective Activation of SMAD3 Signaling Pathway

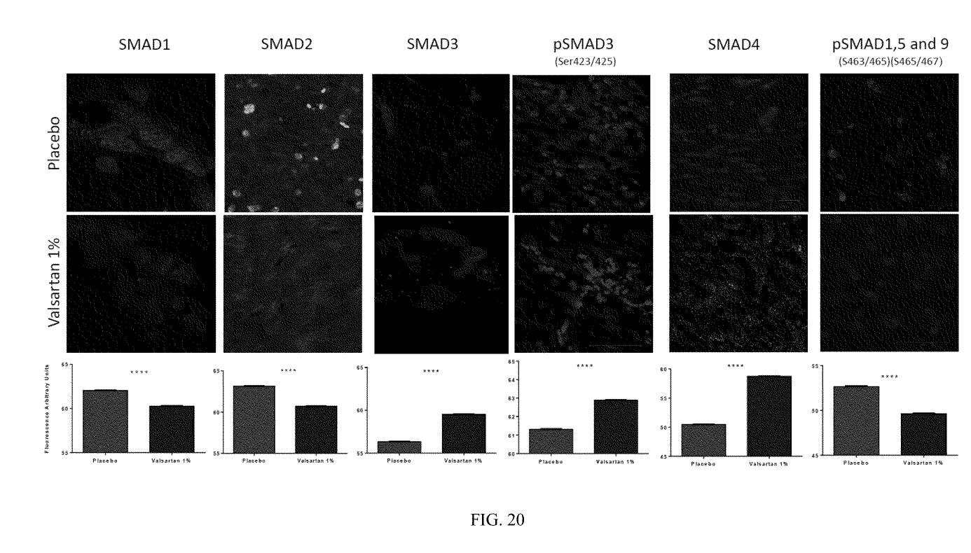

Although not completely characterized, wound healing is greatly influenced by subtle changes of transforming growth factor-beta (TGF-β) superfamily, which is strongly influenced by RAS. TGF-β signaling mediate the phosphorylation of Smad family proteins. Phosphorylated Smads then translocate to the nucleus with the common-mediator (co-Smad) Smad4. Additionally, Smad activity is also regulated by phosphorylation through non-receptor kinases such as p42/44 mitogen activating protein kinase (MAPK) and p38 MAPK. In chronic wounds, failure of TGF-β Smad 2 and 3 phosphorylation and reduction in p42/44 MAPK activity was associated with a slower proliferative rate and impaired healing. RAS has been tightly linked to TGFβ activity but the specific effects of AT₁R on the different Smads during wound healing are not known. To determine the molecular mechanisms by which Valsartan may have influenced wound healing process, changes in Smads (1, 2, 3, 4, 5 and 9) were quantified using immunohistochemistry. These results suggest that topical Valsartan inhibited Smads 1 and 2 but activated Smad 3 (FIG. 20) in wounds of diabetic aged pigs. To examine the impact of increase in Smad3, changes in phosphorylated Smad3 and co-Smad4 were quantified and demonstrated an increase in phosphorylated Smad3 and an increase in co-Smad4 (FIG. 20). Treatment with Valsartan also decreased Smad1, 5, 9 phosphorylation.

### Example 11

### Enriched Mitochondrial, Proliferation and Angiogenesis Markers in Aged Diabetic Pig Wounds Treated with Valsartan

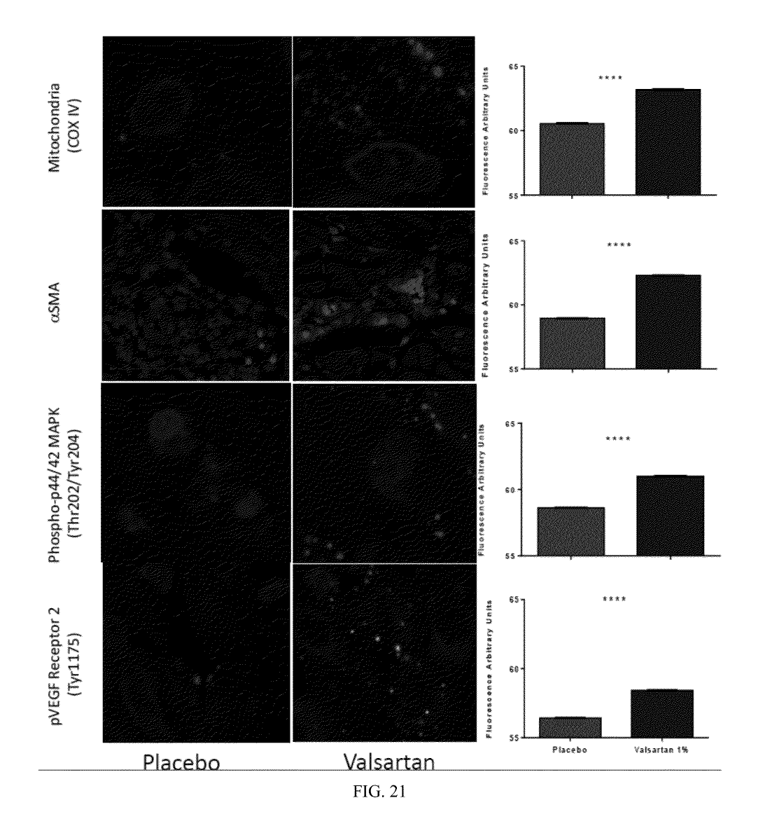

Because topical Valsartan treatment in aged diabetic pig wounds increased Smad3 phosphorylation and increased the rate of granulation tissue formation and re-epithelialization, the effect of Valsartan on factors linked to Smad3 and involved in wound healing was examined. Prior research suggested that in chronic wounds, the decrease in Smad3 was associated with a parallel decrease in MAPK and that exogenous Smad3 administration enhanced alpha-smooth muscle actin (α-SMA), vascular endothelial growth factor (VEGF). In diabetic aged pig wounds, Valsartan enhanced phosphorylation of p42/44 MAPK but not p38MAPK (FIG. 21). An increase in a-SMA and phosphorylated VEGF Receptor 2 was also observed. Finally, the inventors have previously reported the presence of a functional mitochondrial angiotensin system within the mitochondria that played a role in mitochondrial bioenergetics regulation. Other groups reported that the knockout of the AT₁R receptor leads to significant increase in mitochondrial numbers. Evaluation of mitochondrial content in healing wound treated with valsartan revealed a significant increase in the mitochondrial numbers (FIG. 21).

### Example 12

### Valsartan Increases Skin Biomechanical Tensile Strength

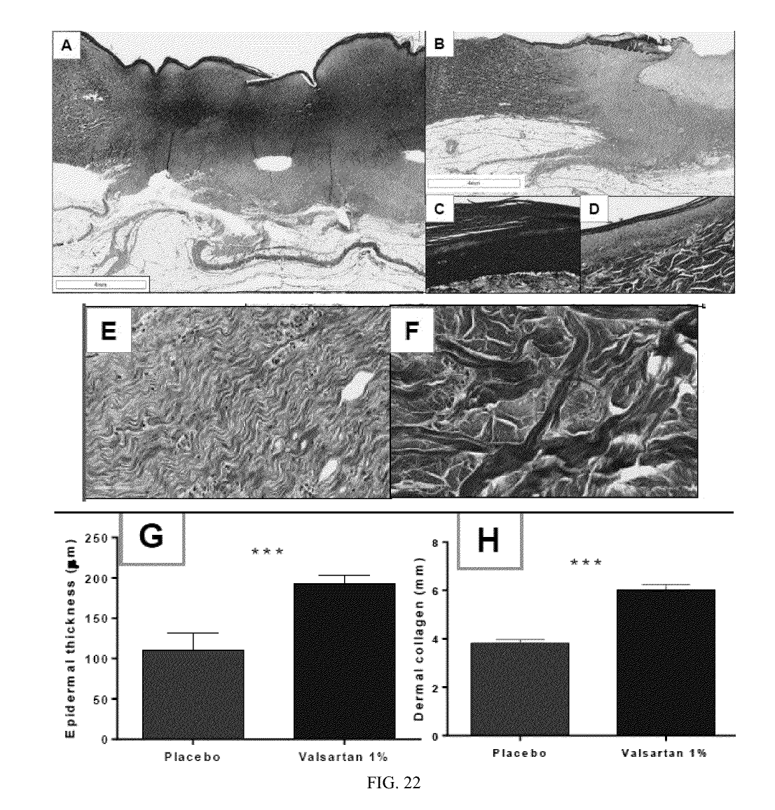

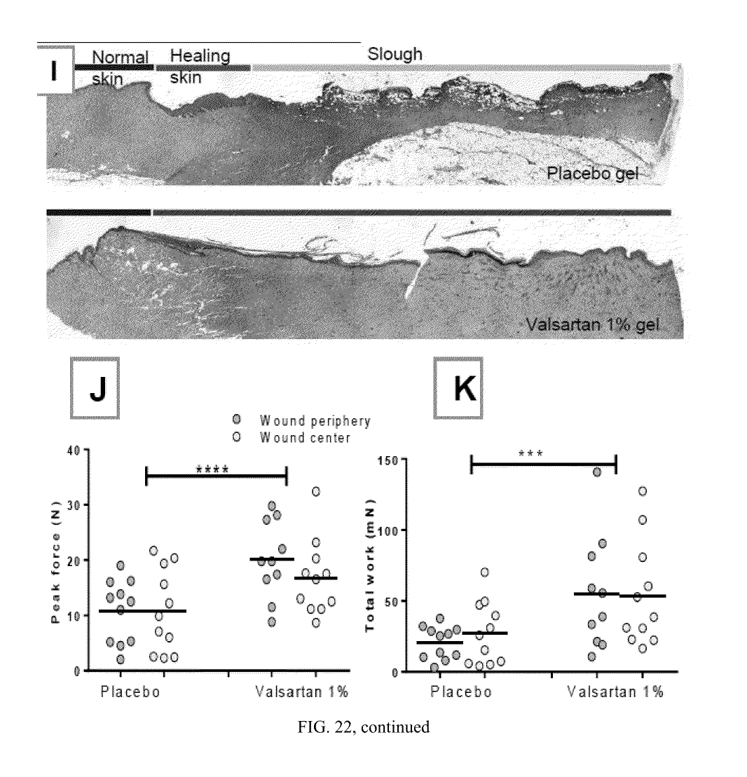

Because faster wound closure and healthy closure may not be synonymous, the impact of Valsartan 1% on the quality of wound repair was also assessed. Using Mason Trichrome (FIG. 22, panels A-H) and H&E stains (FIG. 22, panel I). Collagen content and other histological changes in healing skin were examined. Tensiometry was employed to quantify differences in tensile strength between placebo and valsartan treated wounds. Consistent with the mice data (FIG. 22, panel H), the accelerated wound healing rate observed with Valsartan treatment in aged diabetic pigs was associated with significantly thicker epidermal layer (192±11 μm vs. 91±12 μm; valsartan vs. placebo. P<0.001; FIG. 22, panels C, D, G) and dermal collagen layer (6±0.2 mm vs. 3.9±0.1 mm; valsartan vs. placebo. P<0.001; FIG. 22, panels A, B, H). Ultrastructural analysis revealed more organized collagen fiber arrangement in valsartan treated wound (FIG. 22, panel E) as compared to the coarser and irregular fiber outlines consistent with scar tissue in placebo treated wounds (FIG. 22, panel F). Biomechanically, valsartan treatment yielded significantly stronger healing skin with higher tensile strength (FIG. 22, panels J and K) suggesting more resilience against wound dehiscence and recurrence, a highly relevant concern in diabetic patients.

### Example 13

### Minimal Adverse Effects and Systemic Absorption of Topical Valsartan

There were no observed adverse animal health effects found based on monitoring of body weight, blood pressure, blood glucose levels, or clinical pathology testing (serum chemistry and hematology) during the treatment period. Ultra-Performance Liquid Chromatography was utilized to determine whether or not valsartan was systemically absorbed from the pig wounds treated with topical Valsartan. The results revealed valsartan plasma concentration ranged from 1 nM (below the limit of quantitation) and peaked early in the course of treatment to a maximum of 50 nM. Valsartan was undetectable in blood during later course of treatment (as means of comparison, an expected blood level of Valsartan in a human on oral Valsartan ranges between 4000-5000 nM).

### REFERENCES

(1) Scimeca C L, Bharara M, Fisher T K, Kimbriel H, Mills J L, Armstrong D G. An update on pharmacological interventions for diabetic foot ulcers. Foot Ankle Spec 2010; 3(5):285-302.

(2) Pradhan L, Nabzdyk C, Andersen N D, LoGerfo F W, Veves A. Inflammation and neuropeptides: the connection in diabetic wound healing. Expert Rev Mol Med 2009; 11:e2.

(3) Falanga V. Wound healing and its impairment in the diabetic foot. Lancet 2005; 366(9498):1736-1743.

(4) Stadelmann W K, Digenis A G, Tobin G R. Physiology and healing dynamics of chronic cutaneous wounds. Am J Surg 1998; 176(2A Suppl):26S-38S.

(5) Hao S Y, Ren M, Yang C, Lin D Z, Chen L H, Zhu P et al. Activation of skin renin-angiotensin system in diabetic rats. Endocrine 2011; 39(3):242-250.

(6) Steckelings U M, Wollschlager T, Peters J, Henz B M, Hermes B, Artuc M. Human skin: source of and target organ for angiotensin II. Exp Dermatol 2004; 13(3):148-154.

(7) Yevdokimova N, Podpryatov S. The up-regulation of angiotensin II receptor type 1 and connective tissue growth factor are involved in high-glucose-induced fibronectin production by cultured human dermal fibroblasts. J Dermatol Sci 2007; 47(2):127-139.

(8) Cooper M E. The role of the renin-angiotensin-aldosterone system in diabetes and its vascular complications. Am J Hypertens 2004; 17(11 Pt 2):165-205.

(9) Rodgers K, Verco S, Bolton L, diZerega G. Accelerated healing of diabetic wounds by NorLeu(3)-angiotensin (1-7). Expert Opin Investig Drugs 2011; 20(11):1575-1581.

(10) Abadir P M, Foster D B, Crow M, Cooke C A, Rucker J J, Jain A et al. Identification and characterization of a functional mitochondrial angiotensin system. Proc Natl Acad Sci USA 2011; 108(36):14849-14854.

(11) Burks T N, ndres-Mateos E, Marx R, Mejias R, Van E C, Simmers J L et al. Losartan restores skeletal muscle remodeling and protects against disuse atrophy in sarcopenia. Sci Transl Med 2011; 3(82):82ra37.

(12) Saydam M, Takka S. Bioavailability File: Valsartan. FABAD J. Pharm. Sci. 2007; 32: 185-96.

(13) Siddiqui N, Husain A, Chaudhry L, Alam M S, Mitra M, Bhasin P S. Pharmacological and Pharmaceutical Profile of Valsartan: A Review. Journal of Applied Pharmaceutical Science 2011; 01(04): 12-19.

(14) Marti G, et al. Electroporative transfection with KGF-1 DNA improves wound healing in a diabetic mouse model. Gene Ther. (a Nature journal) 2004; 11(24): 1780-1785.

(15) Dou C, et al. Strengthening the skin with topical delivery of keratinocyte growth factor-1 using a novel DNA plasmid. Mol Ther. 2014; 22(4): 752-761.

(16) Gring CN, Francis G S. A hard look at angiotensin receptor blockers in heart failure. J Am Coll Cardiol 2004; 44: 1841-1846.

(17) Li E C, Heran B S, Wright J M. Angiotensin converting enzyme (ACE) inhibitors versus angiotensin receptor blockers for primary hypertension. Cochrane Database Syst Rev 2014; 8:CD009096.

(18) Zandifar E, Sohrabi B S, Zandifar A, Haghjooy J S. The effect of captopril on impaired wound healing in experimental diabetes. Int J Endocrinol 2012; 2012:785247.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A topical pharmaceutical composition, comprising:
valsartan, in an amount from about 0.2% to about 2.5% by weight of the composition; and a pharmaceutically acceptable carrier material.

2. The pharmaceutical composition of claim 1, wherein valsartan is present in an amount from about 0.5% to about 1.5% by weight of the composition.

3. The pharmaceutical composition of claim 2, wherein valsartan is present in an amount of about 1% by weight of the composition.

4. The pharmaceutical composition of claim 1, wherein the pharmaceutically acceptable carrier material is a cellulosic gel.

5. The pharmaceutical composition of claim 4, wherein the cellulosic gel is present in an amount of about 5% to about 99% of the composition.

6. The pharmaceutical composition of claim 5, wherein the cellulosic gel is present in an amount of about 95% of the composition.

7. The pharmaceutical composition of claim 1, further comprising an aqueous medium.

8. (canceled)

9. The pharmaceutical composition of claim 7, wherein the aqueous medium is present in an amount from about 1% to about 5% by weight of the composition.

10. The pharmaceutical composition of claim 9, wherein the aqueous medium is present in an amount of about 3% by weight of the composition.

11. The pharmaceutical composition of claim 4, wherein the cellulosic gel comprises hydroxypropyl methylcellulose.

12. (canceled)

13. The pharmaceutical composition of claim 1, further comprising:
crospovidone, hydroxypropyl methylcellulose, ferric oxide, magnesium stearate, and titanium dioxide.

14. (canceled)

15. (canceled)

16. The pharmaceutical composition of claim 1, wherein the composition consists essentially of:
valsartan, in an amount from about 0.2% to about 5% by weight of the composition; colloidal silicon dioxide; crospovidone; hydroxypropyl methylcellulose; ferric oxide; magnesium stearate;
microcrystalline cellulose; polyethylene glycol; titanium dioxide; propylene glycol; polypropylene glycol; chlorhexidine gluconate; water; propylene oxide; acetic acid; sodium acetate; and lavender.

17. (canceled)

18. (canceled)

19. The pharmaceutical composition of claim 1, wherein the composition has the formulation of a gel, ointment, cream, bandage, spray, or powder.

20. A method for preparing the pharmaceutical composition of claim 1, comprising the step of:
combining a pharmaceutically acceptable carrier material with valsartan in an amount sufficient to make a composition that is 0.2% to about 2.5% valsartan by weight of the composition.

21-26. (canceled)

27. A method for treating a wound, comprising:
administering to a subject suffering from a wound a therapeutically effective amount of the pharmaceutical composition of claim 1.

28-36. (canceled)

37. A method for treating a cutaneous wound, comprising:
administering to the cutaneous wound in a subject in need thereof a therapeutically effective amount of the pharmaceutical composition of claim 1.

38. The method of claim 37, wherein the cutaneous wound is a chronic wound, a diabetic skin ulcer, or an ulcer associated with aging skin.

39. (canceled)

40. (canceled)

41. The method of claim 27, wherein the wound is a burn, an electrical injury, a radiation injury, a sunburn, a gun shot injury, an explosives injury, a post-surgical wound, a keloid, scar tissue, psoriasis, a superficial dermatologic resurfacing, or a skin lesion due to an inflammatory condition.

42. The method of claim 37, wherein the cutaneous wound is in a tissue associated with an upregulation in angiotensin II type 1 receptors.

43. The method of claim 37, wherein the step of administering is topical administration or buccal administration.

44. (canceled)

45. The method of claim 37, wherein the pharmaceutical composition is administered at least 3 days, at least 4 days, at least 5 days, or at least 6 days after wounding.

46. (canceled)

47. (canceled)
